(19) United States
(12) Patent Application Publication
(10) Pub. No.: US 2003/0003065 A1
(43) Pub. Date: Jan. 2, 2003

(54) Cosmetic compositions exhibiting characteristic first derivative spectral curves and associated methods

(75) Inventors: Karen Kay Kalla, Cincinnati, OH (US); Marcia Lang Canter, Hamilton, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/174,339

(22) Filed: Jun. 18, 2002

Related U.S. Application Data
(60) Provisional application No. 60/299,017, filed on Jun. 18, 2001.

(51) Int. Cl. A61K 7/021; A61K 7/025

(52) U.S. Cl. 424/63; 424/64

(57) ABSTRACT

Cosmetic compositions and cosmetic compositions that have been adapted for delivery to provide applied cosmetic compositions that have a spectrophotometric curve, wherein a first derivative of the spectrophotometric curve comprises: a) a maximum peak in the region of from about 430 nm to about 520 nm occurs at a wavelength not greater than about 480 nm; b) a maximum peak in the region of from about 420 nm to about 650 nm occurs at a wavelength of from about 570 nm to about 630 nm; and c) a minimum valley in the region of from about 520 nm to about 580 nm has a Δ%R/Δλ of less than or equal to about 0.03, wherein R is reflectance and λ is wavelength, and wherein the cosmetic composition comprises a mixture of at least two colorants, wherein a first derivative of a spectrophotometric curve of each of the individual colorants does not exhibit (a), (b) and (c). Methods for providing such compositions comprise adding colorants to a cosmetic composition to provide the composition with a spectrophotometric curve as described.

### CROSS REFERENCE TO RELATED APPLICATIONS

This application claims the benefit of U.S. Provisional Application No. 60/299,017, filed Jun. 18, 2001.

### FIELD OF THE INVENTION

The present invention is directed to cosmetic compositions of improved color as well as cosmetic compositions adapted for application to a substrate to provide applied cosmetic compositions of improved color, for example, improved skin tone color. The cosmetic compositions have a spectrophotometric curve, wherein the spectrophotometric curve has a first derivative that exemplifies maximum and minimum characteristics at specified regions. The present invention is also directed to methods for providing such cosmetic compositions.

### BACKGROUND OF THE INVENTION

Perception of color is determined by the observation of light reflecting across visible wavelengths, specifically within the range of about 400-700 nm. To measure the perceived color of an object, a spectrophotometric curve (also termed “spectral curve”) may be plotted wherein the percentage of reflectance is measured versus the wavelength of light.

After a period of time, the exterior appearance of human skin becomes discolored due to various factors such as environmental exposure, aging and basic human physiology. In an attempt to revitalize and duplicate the youthful yet natural properties of skin, various cosmetic compositions have been developed, including, for example, foundations and concealers. Typically, the cosmetic compositions are applied to the skin to cover imperfections and/or simulate healthy or natural-looking skin. It is therefore desirable to provide cosmetic compositions that exhibit the natural qualities exhibited by skin and particularly which exhibit the color tone of skin, thereby appearing less noticeable and more natural when applied to skin. Despite this desired effect, however, many current cosmetic compositions typically do not substantially match the color and tone of skin. As a result, the human eye is visually able to detect the presence of such cosmetic compositions once they are applied, whereby an unnatural skin look is perceived. Accordingly, there is a desire for providing cosmetic compositions that better match the color of skin and have a natural skin-like appearance. Additionally, it is desirable for these compositions to have enhanced color and to provide sufficient coverage once applied to skin while remaining virtually undetectable to the naked eye, i.e., look like youthful skin.

### SUMMARY OF THE INVENTION

The present invention aims provide novel cosmetic compositions and methods for providing cosmetic compositions that exhibit natural skin tone color. In one embodiment, the present invention is directed to cosmetic compositions having a spectrophotometric curve, wherein a first derivative of the spectrophotometric curve comprises: a) a maximum peak in the region of from about 430 nm to about 520 nm occurs at a wavelength not greater than about 480 nm; b) a maximum peak in the region of from about 420 nm to about 650 nm occurs at a wavelength of from about 570 nm to about 630 nm; and c) a minimum valley in the region of from about 520 nm to about 580 nm has a Δ%R/Δλ of less than or equal to about 0.03, and wherein the cosmetic composition comprises a mixture of at least two colorants, wherein a first derivative of a spectrophotometric curve of each of the individual colorants does not exhibit (a), (b) and (c). Within the present disclosure Δ%R/Δλ represents the change in percent reflectance/change in wavelength as represented in the first derivative of the spectrophotometer curve.

In another embodiment, the present invention is directed to cosmetic compositions adapted for application to a substrate to provide an applied cosmetic composition having a spectrophotometric curve, and comprising at least two colorants. The first derivative of the spectrophotometric curve of the applied cosmetic composition comprises: a) a maximum peak in the region of from about 430 nm to about 520 nm occurs at a wavelength not greater than about 480 nm; b) a maximum peak in the region of from about 420 nm to about 650 nm occurs at a wavelength of from about 570 nm to about 630 nm; and c) a minimum valley in the region of from about 520 nm to about 580 nm has a Δ%R/Δλ of less than or equal to about 0.03, wherein R is reflectance and λ is wavelength, and wherein a first derivative of a spectrophotometric curve of each of the individual colorants does not exhibit (a), (b) and (c).

In a further embodiment, the invention is directed to cosmetic compositions having a skin tone color and a spectrophotometric curve, wherein the first derivative of the spectrophotometric curve comprises: a) a maximum peak in the region of from about 430 nm to about 520 nm occurs at a wavelength not greater than about 480 nm; b) a maximum peak in the region of from about 420 nm to about 650 nm occurs at a wavelength of from about 570 nm to about 630 nm; and c) a minimum valley in the region of from about 520 nm to about 580 nm has a Δ%R/Δλ of less than or equal to about 0.03, wherein R is reflectance and λ is wavelength, and wherein the cosmetic composition comprises a mixture of at least two colorants, wherein a first derivative of a spectrophotometric curve of each of the individual colorants does not exhibit (a), (b) and (c).

In another embodiment, the invention is directed to methods for providing a cosmetic composition with a skin tone color. The methods comprise adding at least two colorants to the cosmetic composition to provide the cosmetic composition with a first derivative of the spectrophotometric curve that comprises: a) a maximum peak in the region of from about 430 nm to about 520 nm occurs at a wavelength not greater than about 480 nm; b) a maximum peak in the region of from about 420 nm to about 650 nm occurs at a wavelength of from about 570 nm to about 630 nm; and c) a minimum valley in the region of from about 520 nm to about 580 nm has Δ%R/Δλ of less than or equal to about 0.03, wherein R is reflectance and λ is wavelength, wherein a first derivative of a spectrophotometric curve of each of the individual colorants does not exhibit (a), (b) and (c).

In still another embodiment, the present invention is directed to methods of providing a colorant mixture for spectrophotometrically matching a cosmetic composition to a target substrate. The methods comprise: a) determining a mixture of colorants adapted to provide a cosmetic composition having a spectrophotometric curve, wherein the first derivative of the spectrophotometric curve comprises: i) a maximum peak in the region of from about 430 nm to about 520 nm occurs at a wavelength not greater than about 480 nm; ii) a maximum peak in the region of from about 420 nm to about 650 nm occurs at a wavelength of from about 570 nm to about 630 nm; and iii) a minimum valley in the region of from about 520 nm to about 580 nm has a Δ%R/Δλ of less than or equal to about 0.03, wherein R is reflectance and λ is wavelength; and b) admixing the colorants to form a mixture; wherein a first derivative of a spectrophotometric curve of each of the individual colorants does not exhibit (a), (b) and (c).

In yet another embodiment, the present invention is directed to methods of providing discrete color domains for spectrophotometrically matching a cosmetic composition to a target substrate. The methods comprise: a) determining a combination of colorants that form discrete color domains adapted to provide a cosmetic composition having a spectrophotometric curve, wherein a first derivative of the spectrophotometric curve comprises: i) a maximum peak in the region of from about 430 nm to about 520 nm occurs at a wavelength not greater than about 480 nm; ii) a maximum peak in the region of from about 420 nm to about 650 nm occurs at a wavelength of from about 570 nm to about 630 nm; and iii) a minimum valley in the region of from about 520 nm to about 580 nm has a Δ%R/Δλ of less than or equal to about 0.03, wherein R is reflectance and λ is wavelength; and b) depositing the colorants into at least two discrete color domains; wherein a first derivative of a spectrophotometric curve of each of the individual colorants does not exhibit (a), (b) and (c).

The cosmetic compositions of the invention exhibit a more natural skin tone color and therefore may be applied without exhibiting a made-up, unnatural look.

These novel features of the present invention will become apparent to those skilled in the art from the following detailed description, which is simply, by way of illustration, various modes contemplated for carrying out the invention. As will be realized, the invention is capable of additional, different obvious aspects, all without departing from the invention. Accordingly, the drawings and specification are illustrative in nature and not restrictive.

### BRIEF DESCRIPTION OF THE DRAWINGS

While the specification concludes with claims particularly pointing out and distinctly claiming the present invention, it is believed that the same will be understood from the following description taken in conjunction with the accompanying drawings in which:

FIG. 1 depicts the spectral curves of skin and a conventional foundation cosmetic product as the percentage of reflectance versus wavelength of light (nm);

FIG. 2 depicts the first derivatives of the spectral curves from FIG. 1 of skin and the conventional foundation cosmetic product as a function of wavelength of light (nm);

FIG. 3 depicts the first derivatives of the spectral curves of skin and two conventional cosmetic foundation products as a function of wavelength of light (nm);

FIG. 4 depicts the spectral curves of skin and a cosmetic composition of the present invention as the percentage of reflectance versus wavelength of light (nm);

FIG. 5 depicts the first derivatives of the spectral curves from FIG. 4 of skin and the cosmetic composition of the present invention as a function of wavelength of light (nm);

### DETAILED DESCRIPTION

The cosmetic compositions and the methods of providing cosmetic compositions according to the present invention provide natural looking cosmetics which exhibit color, as requested by its spectrophotometric curve, that substantially matches the spectrophotometric curve of skin. More particularly, the present invention is directed to cosmetic compositions and methods of providing cosmetic compositions that have a spectrophotometric curve exemplifying maximum and minimum first derivative characteristics at specified regions along the curve to approximate the first derivative of the spectrophotometric curve of skin.

As used herein, “comprising” means that other steps and ingredients can be added. This term encompasses the terms “consisting of” and “consisting essentially of”. The phrase “consisting essentially of” means that the composition may include additional ingredients, but only if the additional ingredients do not materially alter the basic and novel characteristics of the claimed compositions or methods.

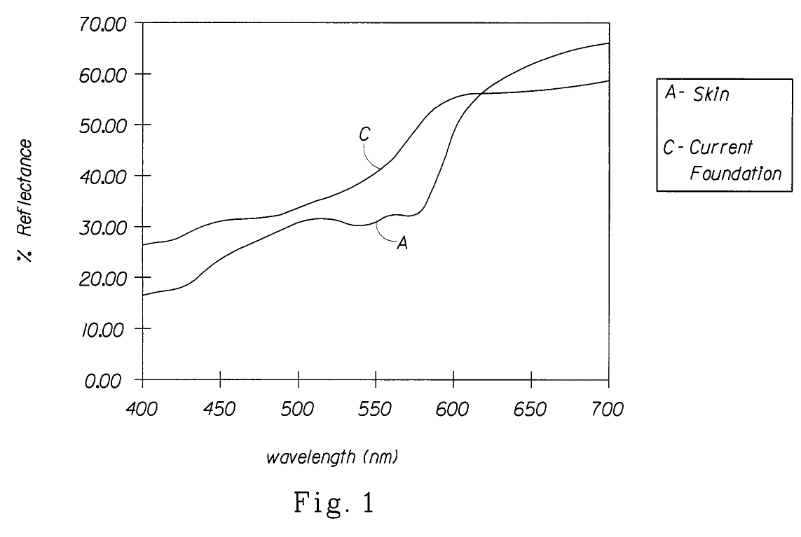

As used herein, both “spectrophotometric curve” and “spectral curve” refer generally to a plotted curve displaying an ordinate value of relative reflectance versus an abscissa value of wavelength of light, typically within the visual range of 400-700 nm. It is generally known that various perceived colors can be compared, particularly by measuring and plotting the reflectance of light across visible wavelengths to produce a spectral curve. Once the spectral curves of the perceived colors are produced, it is then possible to compare the measured curves for any distinguishing color characteristics which are exhibited by the individual colors. Upon analyzing the spectral curve of human skin, and the first derivative thereof, the inventors of the present invention believe, without being limited by theory, that all skin types (no matter the age or ethnicity of the individual) exhibit common color characteristics. As a result, cosmetic compositions can be spectrally compared to a human skin sample to determine whether these common color characteristics are present within the composition. For instance, FIG. 1 depicts a graph comparing the spectral curves of human skin and a conventional cosmetic composition comprising a foundation within the region of visible light (400 nm to 700 nm). As can be seen in FIG. 1, the spectral curve of skin (curve A) and the spectral curve of the foundation (curve C) appear somewhat similar in shape; for instance, both curves exhibit plotted lines generally sloping upward from 400 to 700 nm. However, the present inventors have determined that the general resemblance in shape of spectral curves is not sufficient to predict how a color is ultimately perceived.

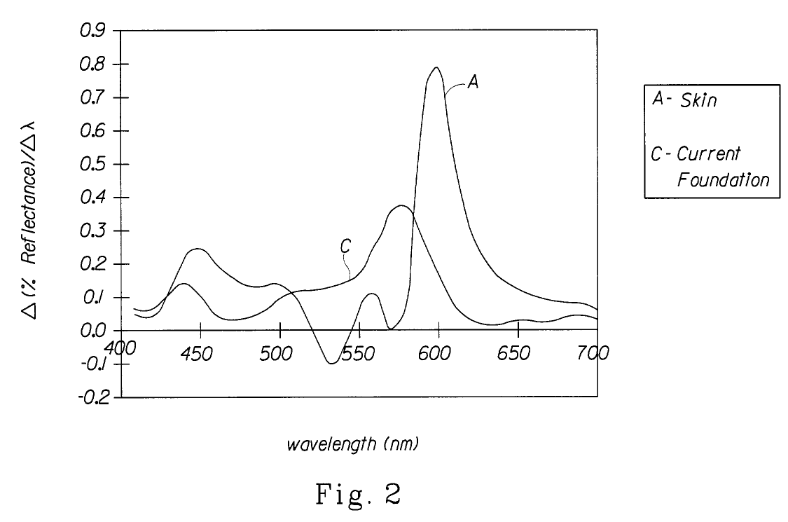

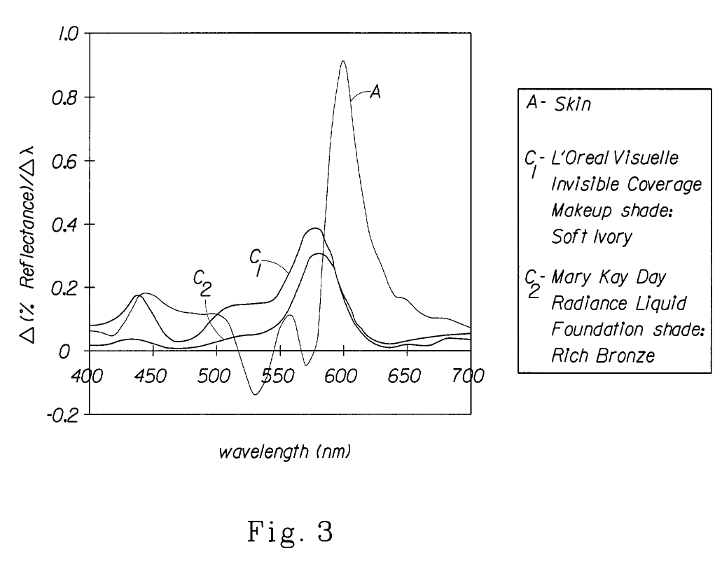

For instance, FIG. 1 indicates that conventional foundations exhibit a spectral curve that appears to generally match or mimic the shape of skin's spectral curve. FIG. 2 depicts the first derivatives of the spectral curves of FIG. 1 and more clearly exemplifies specific color characteristics along the curves. A comparison of the first derivative curves in FIG. 2 demonstrates that the first derivative curve of the foundation (curve C) is substantially different from that of skin. As depicted by FIG. 3, even when a wide range of foundations are compared to skin, current cosmetics generally exhibit first derivative curves that do not substantially match or mimic the first derivative spectral curve of skin. More particularly, when compared to the first derivative of the spectral curve of skin (line A), both of the curves for conventional foundations (Curve C1 exhibiting a light, Caucasian type foundation and Curve C2 exhibiting a dark, African-American type foundation) fail to mimic or match the minimum and maximum characteristics exemplified by the curve of skin within the visible light spectrum at the first derivative level. Accordingly, these unmatched cosmetic compositions fail to exhibit all of the color properties exhibited by human skin and thereby appear less natural and/or dull.

As mentioned above, Applicants, without being limited by theory, believe that all human skin types, no matter the age or ethnicity of the individual, typically exhibit various common characteristics at specified regions along a spectral curve and the first derivative thereof. Moreover, it is believed that these common characteristics must be substantially matched or mimicked by a cosmetic composition to achieve the youthful, clear, natural look exhibited by skin. In determining a substantial match, it is believed that these common characteristics are more clearly defined at the first derivative level of the spectral curve. Accordingly, the first derivative of the spectral curve of skin is used to characterize cosmetic compositions with a perceived color interpretation similar to human skin by substantially matching or mimicking the first derivative of the spectral curve of the cosmetic composition with the first derivative of the spectral curve of skin.

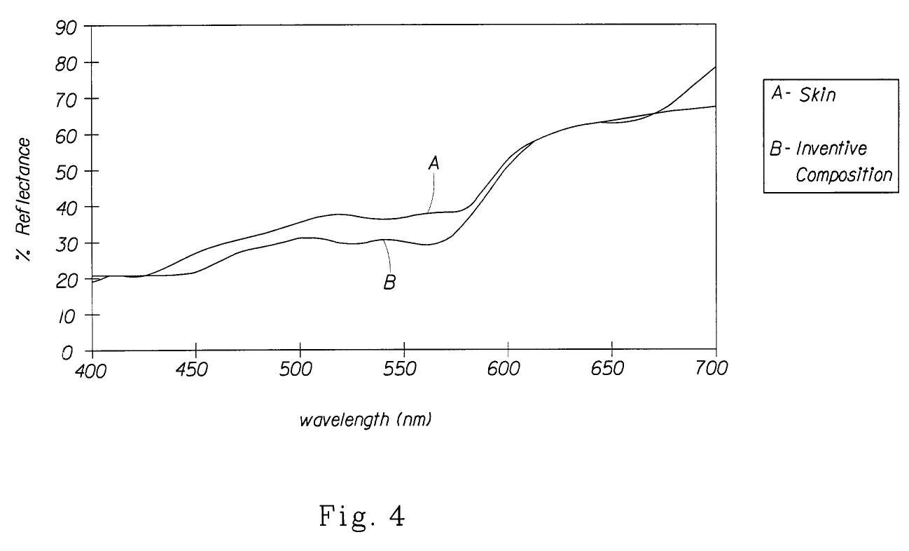

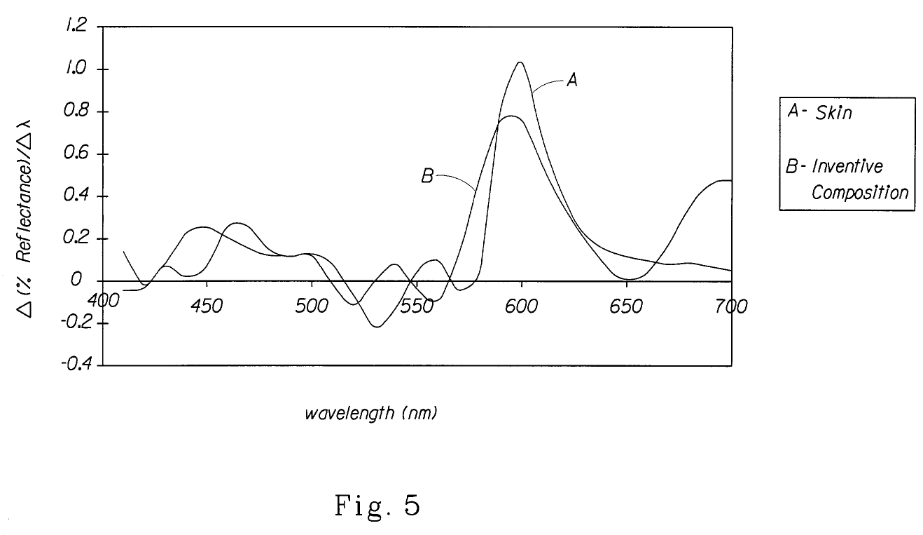

Thus, the cosmetic compositions of the present invention have a spectral curve wherein the first derivative thereof exemplifies both maximum and minimum characteristics similar to the first derivative of the spectral curve of skin. As a result, these cosmetics closely resemble the perceived color of human skin and thereby appear natural when applied to skin. For instance, as depicted by FIG. 4, the spectral curve of an inventive composition according to the present invention (curve B) substantially resembles the spectral curve of human skin (curve A). More particularly, the spectral curves of both skin (A) and the inventive composition (B) are more closely matched. Moreover, the substantial similarity between the first derivative of the spectral curve of skin and that of the inventive cosmetic composition is depicted in FIG. 5. As seen in FIG. 5, the cosmetic compositions according to the present invention exemplify maximum and minimum first derivative characteristics at specified regions along the curve similar to that of skin's spectral curve. For instance, the cosmetic composition (curve B) of the present invention has a maximum peak similar to that of skin (curve A) within the region of from about 430 nm to about 520 nm, namely occurring at a wavelength of not greater than about 480 nm. Additionally, the cosmetic composition of the present invention (curve B) exhibits a maximum peak within the region of from about 420 nm to about 650 nm, namely occurring at a wavelength of from about 570 nm to about 630 nm, similarly to that of skin (curve A). Lastly, the cosmetic composition of the invention exhibits minimum valley in the region of from about 520 nm to about 580 nm having a Δ%R/Δλ of less than or equal to about 0.03 (curve B), similarly to that of (curve A), wherein R=reflectance and λ=wavelength. Preferably, Δ%R/Δλ is less than or equal to about 0.015, and most preferably, less than or equal to about 0.

In one embodiment of the present invention, a cosmetic composition has a spectrophotometric curve, wherein the spectrophotometric curve has a first derivative exhibiting a maximum peak in the region of from about 430 nm to about 520 nm occurs at a wavelength not greater than about 480 nm. Moreover, the first derivative of spectrophotometric curve additionally has a maximum peak within the region of from about 420 nm to about 650 nm that occurs at a wavelength of from about 570 nm to about 630 nm, and a minimum valley in the region of from about 520 nm to about 580 nm has Δ%R/Δλ of less than or equal to about 0.03, wherein R is reflectance and λ is wavelength. Preferably, Δ%R/Δλ is less than or equal to about 0.015, and most preferably, less than or equal to about 0.

The cosmetic compositions of the present invention may be provided with the spectrophotometric curve as described by a variety of means and methods and using various types and combinations of two or more colorants. As will be described in further detail below, in one embodiment, the cosmetic compositions of the invention may be provided with the desired spectrophotometric curve by using a physical mixture of two or more colorants, wherein, once mixed, the colorants are no longer discernible or distinguishable from one another. In an alternate embodiment, the cosmetic compositions of the invention may be provided with the desired spectrophotometric curve by use of at least two discrete color domains, each of which contains a colorant, and which are not discernible to the naked eye, i.e., at a macroscopic level, but can be distinguished from one another under magnification.

Color Domains

The cosmetic compositions of the present invention may comprise two or more discrete color domains that provide the desired spectrophotometric curve of the cosmetic composition that is substantially similar to the spectrophotometric curve of skin. As used herein, “cosmetic compositions” refers to a prepackaged formulation of the cosmetic product as well as to a formulation that may be adapted for delivery to a substrate to provide an applied cosmetic product. Also, a “discrete color domain” generally refers to a separate and discrete colored region of space within the cosmetic composition and/or upon delivery to a substrate, with each separate and discrete color domain individually comprising a color expression within a specified size area. At least two discrete color domains are included in the cosmetic compositions to provide the desired spectrophotometric curve. A plurality of discrete color domains may also be combined in the cosmetic compositions. Suitable color domains within the cosmetic compositions are those which may be arranged in such a manner that each discrete domain is capable of separately producing an individual color expression while remaining individually undetected by the human eye at a macroscopic level, i.e., to a casual observer. The two or more color domains in a cosmetic composition cooperate to provide the composition with the desired spectrophotometric curve.

The discrete color domains will typically have of an average size of from about 5 μm to about 500 μm, although one skilled in the art will appreciate that discrete color domains of sizes outside of this range may also be suitable for providing the desired spectrophotometric curve. In a more specific embodiment, the discrete color domains have an average size of from about 10 μm to about 300 μm, preferably from about 10 μm to about 200 μm, more preferably from about 10 μm to about 100 μm, and in a further embodiment have an average size of from about 10 μm to about 70 μm.

In one embodiment, the cosmetic compositions of the present invention comprise two or more color domains, wherein each color domain comprises at least one colorant, respectively. Alternatively, the cosmetic composition may comprise at least two color domains wherein at least one of the color domains comprises a mixture of colorants. As used herein, “mixture” is meant to include a simple combination of materials and any compounds that may result from their combination. As used herein, “colorants” generally refer to a dye, pigment, lake or other agent used to impart a color expression to a material. Colorants employed in the present invention, if solid or semisolid, typically have an average size of from about 0.01 μm to about 500 μm, preferably from about 0.1 μm to about 300 μm, more preferably from about 0.01 μm to about 100 μm, even more preferably from about 0.01 μm to about 70 μm, and most preferably, from about 0.01 μm to about 20 μm. It should be understood that each discrete color domain can consist wholly of a single colorant, or a mixture of two or more colorants, and can optionally further include a carrier for the one or more colorants. It should also be understood that a first derivative of the spectrophotometric curves of each of the individual colorants included within the cosmetic compositions according to the present invention does not exhibit the first derivative characteristics of the spectrophotometric curve. As will be explained in more detail below, the entire or overall cosmetic composition itself, which includes the colorants, will express the spectrophotometric characteristics at the first derivative level.

Some of the colorants which can be used herein include, but are not limited to, D&C Yellow No. 7, D&C Red No. 36, FD&C Red No. 4, D&C Orange No. 4, D&C Red No. 6, D&C Red No. 34, FD&C Yellow No. 6, D&C Red No. 33, FD&C Yellow No. 5, D&C Brown No. 1, D&C Red No. 17, FD&C Green No. 3, D&C Blue No. 4, D&C Yellow No. 8, D&C Orange No. 5, D&C Red No. 22, D&C Red No. 21, D&C Red No. 28, D&C Orange No. 11, D&C Yellow No. 10, D&C Violet No. 2, Ext. D&C Violet No. 2, D&C Green No. 6, D&C Green No. 5, D&C Red No. 30, D&C Green No. 8, D&C Red No. 7, FD&C Blue No. 1, D&C Yellow No. 7, D&C Red No. 27, D&C Orange No. 10, D&C Red No. 31, FD&C Red No. 40, D&C Yellow No. 11, Annatto extract, β carotene, guanine, carmine, aluminum powder, ultramarines, bismuth oxychloride, chromium oxide green, chromium hydroxide green, iron oxides, ferric ferrocyanide, manganese violet, titanium dioxide, zinc oxide, caramel coloring, mica, ferric ammonium ferrocyanide, dihydroxyacetone, guaiazulene, pyrophyllite, bronze powder, copper powder, aluminum stearate, calcium stearate, lactofavin, magnesium stearate, zinc stearate, capsanthin/capsorubin, bentonite, barium sulfate, calcium carbonate, calcium sulfate, carbon black, magnesium carbonate, colored silica, Cl 10020, Cl 11680, Cl 15630, Cl 15865, Cl 16185, Cl 16255, Cl 16255, Cl 45430, Cl 69825, Cl 73000, 73015, Cl 74160, Cl 75100, Cl 77002, Cl 77346, Cl 77480. Additionally, lakes or composites of these colorants may also be used. Preferred colorants are selected from the group consisting of Yellow 5, Yellow 10, Red 7 Ca Lake, red iron oxide, titanium dioxide, and combinations thereof.

One skilled in the art will appreciate that the cosmetic compositions comprising two or more color domains may comprise any combination of the various types of color domains described herein. Thus, for example, a first color domain containing a single colorant may be used together with a second color domain containing a mixture of colorants, and/or a third color domain containing one or more colorants and a carrier. Alternatively, similar types of color domains may be employed together.

To identify mixtures or combinations of color domains that provide the cosmetic compositions with the desired spectrophotometric curve, trial and error, combinatorial experiments and mathematical models such as the Neugebauer thoery, Murray-Davis equation, and Yule-Nielsen equation may be utilized. See Zeng, Huanzao, Chin, Bob, “A Printer Model for Color Printing,” Annual Conference—Society for Imaging Science and Technology, Volume 50: p. 284-288, (1997).

Physical Mixtures

The cosmetic compositions of this invention may alternatively be provided with the desired spectrophotometric curve by use of a physical mixture of at least two colorants, including solid and semi-solid colorants as well as soluble dyes. As used herein, “physical mixture” is the product of admixing or combining two or more colorants wherein, once mixed, the colorants are no longer individually discernible or distinguishable from one another. Once combined, the colorants together provide the composition with a spectral curve exhibiting the desired maximum and minimum first derivative characteristics at the specified wavelength regions. Solid or semi-solid colorants to be physically admixed typically have an average particle size of from about 0.01 μm to about 500 μm. Additional preferred average particle sizes for the colorant admixtures are from about 0.01 μm to about 300 μm, preferably from about 0.01 μm to about 100 μm, more preferably from about 0.01 μm to about 70 μm, and in a further embodiment from about 0.01 μm to about 20 μm. It should be understood, however, that colorants having an average particle size outside of these ranges may also be used. Suitable colorants include, but are not limited to, those listed above in the Color Domains section. Preferred colorants for the physical mixtures are selected from the group consisting of Yellow 5, Yellow 10, Carmine, Red 33, Green 5, Yellow 8, and combinations thereof.

To identify colorants suitable for physical mixtures to provide the desired spectrophotometric curve to the cosmetic compositions, trial and error, combinatorial experiments and known mathematical models such as the Kubelka-Munk equation may be utilized. See Judd, Deane B., Wyszecki, Günter, Color in Business, Science and Industry”, 2^{nd} Edition, John Wiley and Sons, Inc., New York, 1963.p. 387-426.

Carrier

The compositions of the present invention may comprise a safe and effective amount of a dermatologically acceptable carrier within which a colorant, physical mixtures, and optional other ingredients are incorporated to enable such materials to be delivered to the skin in an appropriate concentration. As used herein, “carrier” generally refers to one or more compatible solid or liquid fillers, diluents, extenders, substrates, mechanisms or the like which carries, transports, conducts or conveys the colorant. The carrier can thus act as a diluent, dispersant, solvent, or the like for a particulate material. In one embodiment, a color domain may comprise a colorant and a carrier.

The carrier may contain one or more dermatologically acceptable solid, semi-solid or liquid fillers, diluents, solvents, extenders and the like. The carrier may be solid, semi-solid or liquid. The carrier can itself be inert or it can possess dermatological benefits of its own. Concentrations of the carrier can vary with the carrier selected and the intended concentrations of the essential and optional components.

Suitable carriers include conventional or otherwise known carriers that are dermatologically acceptable. The carrier should also be physically and chemically compatible with the essential components described herein, and should not unduly impair stability, efficacy or other use benefits associated with the compositions of the present invention. Preferred components of the compositions of this invention should be capable of being commingled in a manner such that there is no interaction that would substantially reduce the efficacy of the composition under ordinary use situations.

The colorants of the present compositions may be formed by incorporation of the colorants into the carrier that results in the combined carrier/colorant having color. For the purposes of this invention, “incorporation” is used to described any method capable of accomplishing a combined colorant/carrier composition. For example, encapsulation, entrapment, dispersion, solvation or the like via any suitable method can be utilized such that it meets the definition of the invention described herein. Combined colorant/carrier variations may be solid particles or mixtures of solids and liquids.

The type of carrier utilized in a particular product in accordance with the present invention may depend on the type and/or physical form of the product desired. More particularly, the cosmetic compositions of the invention may be of a wide variety of product forms. These include, but are not limited to, lotions, creams, gels, sticks, sprays, ointments, pastes, mousses and cosmetics (e.g., solid, semi-solid, or liquid make-up, including foundations, eye-makeup, pigmented or non-pigmented lip treatments, e.g., lipsticks, and the like). These product forms may comprise several types of carriers including, but not limited to, solutions, aerosols, emulsions, gels, solids, and liposomes. Suitable personal care product forms are disclosed in U.S. Ser. No. 09/502395, filed Feb. 11, 2000 (Jakubovic et al.); 09/544789, 09/544788, 09/544783, 09/54490, 09/544791, all filed Apr. 7, 2000 (Robinson et al.); 09/583616, 09/629765 09/628630, 09,629734, all filed Jul. 31, 2000 (Yen et al.); 09/249217 (Feb. 12, 1999, 09/502395 (filed Feb. 11, 2000), U.S. Pat. Nos. 6,071,503, 6,139,823, 6,019,962, 6,106,820, 6,017,552, 6,013,269, and 6,001,373, each incorporated by reference in its entirety.

These product forms may comprise several types of color domain carriers including, but not limited to, colloids, polymer encapsulates or solid carriers. Specific embodiments of the present invention also include cosmetic compositions comprising at least two color domains wherein at least one of the color domains comprises a mixture of colorants and a carrier.

Aerosols according to the subject invention can be formed by adding a propellant to a solution such as described above. Exemplary propellants include chloro-fluorinated lower molecular weight hydrocarbons. Additional propellants that are useful herein are described in Sagarin, Cosmetics Science and Technology, 2nd Edition, Vol. 2, pp. 443-465 (1972), incorporated herein by reference. Aerosols are typically applied to the skin as a spray-on product.

Carriers of the present invention may contain a dermatologically acceptable, hydrophilic diluent. As used herein, “diluent” includes materials in which the particulate material can be dispersed, dissolved, or otherwise incorporated. Nonlimiting examples of hydrophilic diluents are water, organic hydrophilic diluents such as lower monovalent alcohols (e.g., C₁-C₄) and low molecular weight glycols and polyols, including propylene glycol, polyethylene glycol (e.g., Molecular Weight 200-600 g/mole), polypropylene glycol (e.g., Molecular Weight 425-2025 g/mole), glycerol, butylene glycol, 1,2,4-butanetriol, sorbitol esters, 1,2,6-hexanetriol, ethanol, isopropanol, sorbitol esters, butanediol, ether propanol, ethoxylated ethers, propoxylated ethers and combinations thereof.

Preferred carriers comprise an emulsion comprising a hydrophilic phase comprising a hydrophilic component, e.g., water or other hydrophilic diluent, and a hydrophobic phase comprising a hydrophobic component, e.g., a lipid, oil or oily material. As well known to one skilled in the art, the hydrophilic phase will be dispersed in the hydrophobic phase, or vice versa, to form respectively hydrophilic or hydrophobic dispersed and continuous phases, depending on the composition ingredients. In emulsion technology, the term “dispersed phase” is a term well-known to one skilled in the art which means that the phase exists as small particles or droplets that are suspended in and surrounded by a continuous phase. The dispersed phase is also known as the internal or discontinuous phase. The emulsion may be or comprise (e.g., in a triple or other multi-phase emulsion) an oil-in-water emulsion or a water-in-oil emulsion such as a water-in-silicone emulsion. Oil-in-water emulsions typically comprise from about 1% to about 50% (preferably about 1% to about 30%) of the dispersed hydrophobic phase and from about 1% to about 98% (preferably from about 40% to about 90%) of the continuous hydrophilic phase; water-in-oil emulsions typically comprise from about 1% to about 98% (preferably from about 40% to about 90%) of the dispersed hydrophilic phase and from about 1% to about 50% (preferably about 1% to about 30%) of the continuous hydrophobic phase. The emulsion may also comprise a gel network, such as described in G. M. Eccleston, Application of Emulsion Stability Theories to Mobile and Semisolid O/W Emulsions, Cosmetics & Toiletries, Vol. 101, November 1996, pp. 73-92, incorporated herein by reference. Additional emulsions that are suitable as carriers for the present invention included water-in-elastomer emulsions as well as elastomer-in-water emulsions.

Preferred carriers comprise a combination of emulsifying and non-emulsifying crosslinked organopolysiloxane elastomer. The term “non-emulsifying,” as used herein, defines crosslinked organopolysiloxane elastomer from which polyoxyalkylene units are absent. The term “emulsifying,” as used herein, means crosslinked organopolysiloxane elastomer having at least one polyoxyalkylene unit. Emulsifying crosslinked organopolysiloxane elastomer can notably be chosen from the crosslinked polymers described in U.S. Pat. No. 5,412,004 (issued May 2, 1995); U.S. Pat. No. 5,837,793 (issued Nov. 17, 1998); and U.S. Pat. No. 5,811,487 (issued Sep. 22, 1998), all of which are herein incorporated by reference in their entirety. Particularly useful emulsifying elastomers are polyoxyalkylene modified elastomers formed from divinyl compounds, particularly siloxane polymers with at least two free vinyl groups, reacting with Si—H linkages on a polysiloxane backbone. Preferably, the elastomers are dimethyl polysiloxanes crosslinked by Si—H sites on a molecularly spherical MQ resin.

Preferred carriers may also comprise a colloidal dispersion. A colloidal dispersion, by definition, is a two phase system that comprises a dispersed phase and a dispersion medium. The state of the dispersed phase (gas, solid, or liquid) in the dispersion medium defines the system as a foam, suspension, or emulsion. The particle size of the dispersed phase further defines the system as a colloidal dispersion versus suspension and microemulsion versus macroemulsion.

Carriers can comprise polymeric materials. These materials can be solid or semi-solid particles, film formers or gelling agents. The polymers herein can be selected from nonionic, ionic (anionic or cationic), and amphoteric (including zwitterionic) polymers. The polymers can have a variety of architectures such as linear, branched, block, graft, star, dendrimer, comb or the like. The distribution of monomers may be controlled, statistical, or random. The polymers may be soluble or crosslinked (thermoplastic or thermoset). The polymers may be organic or inorganic or a combination thereof. The polymers may be blended or mixed with other polymers. The polymers may be modified with additional ingredients known to one skilled in the art including but not limited to plasticizers, fillers, oligomers, surface modifiying agents, UV protectants, opacifiers, refractive index modifiers, and processing aids. Suitable polymers include any known in the art, such as, polyacrylics, polymethacrylics, polystyrenes, polysiloxanes, polyesters, polyurethanes, polyureas, poly amides, urethane-acrylic copolymers, styrene-acrylic copolymers, siloxane-urethane copolymers, silicone-acrylic copolymers, silicone grafted polymers, silicone block copolymers, polyolefins, vinyl esters, vinyl ethers, polyvinyl pyrrolidones or other vinyl heterocycles, cellulosic polymers and mixtures thereof. As used herein, a “polyacrylic” includes polyacrylates, polyacrylics, or polyacrylamides, and “polymethacrylic” includes polymethacrylates, polymethacrylics, or polymethacrylamides. Styrene-acrylic copolymers include copolymers of styrene with acrylate, acrylic, acrylamide, methacrylate, methacrylic, and/or methacrylamide monomers. Additional suitable polymers can be found in polymer chemistry and science books such as The Principles of Polymerization by George Odian, Fundamentals of Polymer Science by Paul C. Painter and Michael M. Coleman, Polymer Syntheses volumes 1-3 by Stanley R. Sandle and Wolf Karo. Some additional polymeric particles include particles of polyamide and more specifically Nylon 12, especially such as those sold by Atochem under the name Orgasol 2002D Nat C05, polystyerene microspheres such as for example those sold by Dyno Particles under the name Dynospheres, ethylene acrylate copolymer sold by Kobo under the name FloBead EA209 and mixtures thereof, Ronasphere LDP from Kobo Inc. Additional organic polymeric particles can be chosen from the methylsilsesquioxane resin microspheres such as for example those sold by Toshiba silicone under the name Tospearl 145A; microspheres of polymethylmethacrylates such as those sold by Seppic under the name Micropearl M 100; the spherical particles of crosslinked polydimethylsiloxanes, especially such as those sold by Dow Corning Toray Silicone under the name Trefil E 506C or Trefil E 505C. Additional polymeric film formers include polyquaternium materials, e.g., Luviquat series from BASF, chitosan and chitosan based materials including cellulose and cellulose-based materials.

Carriers can also be inorganic spherical, non-spherical or platelet particles such as silicas, silicates, carbonates, mica, sericite, talc, titanium dioxide, barium sulfate, clays, zinc oxide, alumina, aluminum benzoate, calcium carbonate, laking agents (aluminum, barium, sodium, potassium, calcium, strontium, zirconium) and combinations thereof.

Carriers can also be associated structures such as liquid crystals, vesicles.

Carriers can also contain solidifying or gelling agents. Suitable solidifying agents include waxy materials such as candelilla, carnauba waxes, beeswax, spermaceti, carnauba, baysberry, montan, ozokerite, ceresin, paraffin, synthetic waxes such as Fisher-Tropsch waxes, silicone waxes (e.g., DC 2503 from Dow Corning), microcrystalline waxes and the like; soaps, such as the sodium and potassium salts of higher fatty acids, i.e., acids having from 12 to 22 carbon atoms; amides of higher fatty acids; higher fatty acid amides of alkylolamines; dibenzaldehyde-monosorbitol acetals; alkali metal and alkaline earth metal salts of the acetates, propionates and lactates; and mixtures thereof. Also useful are polymeric materials such as, locust bean gum, sodium alginate, sodium caseinate, egg albumin, gelatin agar, carrageenin gum sodium alginate, xanthan gum, quince seed extract, tragacanth gum, starch, chemically modified starches and the like, semi-synthetic polymeric materials such as cellulose ethers (e.g. hydroxyethyl cellulose, methyl cellulose, hydroxypropyl cellulose, carboxymethyl cellulose, hydroxy propylmethyl cellulose), polyvinylpyrrolidone, polyvinylalcohol, guar gum, hydroxypropyl guar gum, soluble starch, cationic celluloses, cationic guars and the like and synthetic polymeric materials such as carboxyvinyl polymers, polyvinylpyrrolidone, polyvinyl alcohol polyacrylic acid polymers, polymethacrylic acid polymers, polyvinyl acetate polymers, polyvinyl chloride polymers, polyvinylidene chloride polymers and the like. Inorganic thickeners may also be used such as aluminum silicates, such as, for example, bentonites, or a mixture of polyethylene glycol and polyethylene glycol stearate or distearate. Naturally occurring polymers or biopolymers and their use are further described in European Application No. 522624, to Dunphy et al. Additional examples of naturally occurring polymers or biopolymers can be found in the Cosmetic Bench Reference, pp. 1.40-1.42, herein incorporated by reference.

Also useful herein are hydrophilic gelling agents such as the acrylic acid/ethyl acrylate copolymers and the carboxyvinyl polymers sold by the B. F. Goodrich Company under the trademark of Carbopol Registered TM resins. These resins consist essentially of a colloidally water-soluble polyalkenyl polyether crosslinked polymer of acrylic acid crosslinked with from 0.75% to 2.00% of a crosslinking agent such as polyallyl sucrose or polyallyl pentaerythritol. Examples include Carbopol 934, Carbopol 940, Carbopol 950, Carbopol 980, Carbopol 951 and Carbopol 981. Carbopol 934 is a water-soluble polymer of acrylic acid crosslinked with about 1% of a polyallyl ether of sucrose having an average of about 5.8 allyl groups for each sucrose molecule. Also suitable for use herein are carbomers sold under the Trade Name “Carbopol Ultrez 10, Carbopol ETD2020, Carbopol 1382, Carbopol 1342 and Pemulen TR-1 (CTFA Designation: Acrylates/10-30 Alkyl Acrylate Crosspolymer). Combinations of the above polymers are also useful herein. Other gelling agents suitable for use herein include oleogels such as trihydroxystearin.

Hydrophobically modified celluloses are also suitable for use herein. These celluloses are described in detail in U.S. Pat. Nos. 4,228,277 and 5,104,646, both of which are herein incorporated by reference in their entirety.

Additional examples of suitable gelling agents or gellants can be found in the Cosmetic Bench Reference, p. 1.27, herein incorporated by reference. Further examples of suitable solidifying agents disclosed in the following references, all of which are incorporated by reference herein: U.S. Pat. No. 4,151,272, Geary, et al., issued Apr. 24, 1979; U.S. Pat. No. 4,229,432, Geria, issued Oct. 21, 1980; and U.S. Pat. No. 4,280,994, Turney, issued Jul. 28, 1981; “The Chemistry and Technology of Waxes”, A. H. Warth, 2nd Edition, reprinted in 1960, Reinhold Publishing Corporation, pp 391-393 and 421; “The Petroleum Chemicals Industry”, R. F. Goldstein and A. L. Waddeam, 3rd Edition (1967), E & F. N. Span Ltd., pp 33-40; “The Chemistry and Manufacture of Cosmetics”, M. G. DeNavarre, 2nd edition (1970), Van Nostrand & Company, pp 354-376; and in “Encyclopedia of Chemical Technology:, Vol. 24, Kirk-Othmer, 3rd Edition (1979) pp 466-481; U.S. Pat. No. 4,126,679, Davy, et al., issued Nov. 21, 1978; European Patent Specification No. 117,070, May, published Aug. 29, 1984; U.S. Pat. No. 2,900,306, Slater, issued Aug. 18, 1959; U.S. Pat. No. 3,255,082, Barton, issued Jun. 7, 1966; U.S. Pat. No. 4,137,306, Rubino, et al., issued Jan. 30, 1979; U.S. Pat. No. 4,154,816, Roehl, et al., issued May 15, 1979; U.S. Pat. No. 4,226,889, Yuhas, issued Oct. 7, 1980; U.S. Pat. No. 4,346,079, Roehl, issued Aug. 24, 1982; U.S. Pat. No. 4,383,988, Teng, et al., issued May 17, 1983; European Patent Specification No. 107,330, Luebbe, et al., published May 2, 1984; European Patent Specification No. 24,365 Sampson, et al., published Mar. 4, 1981; and U.S. patent application Ser. No. 630,790, DiPietro, filed Jul. 13, 1984.

Delivery Means

As mentioned herein relative to the phrase “cosmetic compositions”, the cosmetic compositions of the present invention may be adapted for delivery to a defined surface via a delivery mechanism. For example, suitable delivery mechanisms include, but are not limited to, colloidal systems, delivery substrates, i.e., applicator pads, sponges, open cell foams, brushes, packages, spraying devices and printing devices. More specifically, delivery devices such as electrostatic spraying devices, air-spraying devices, ink jet print heads, lithographic devices or stamp pads are suitable for use herein.

In certain embodiments, the cosmetic compositions of the invention are adapted for application to a substrate to provide an applied cosmetic composition having a spectrophotometric curve whose first derivative exhibits desirable characteristics. In such embodiments, the cosmetic composition adapted for application is not required to itself have a spectrophotometric curve whose first derivative exhibits the desired maximum and minimum characteristics. Rather, it is only required that the composition is adapted to provide, for example, via a delivery mechanism, an applied cosmetic composition whose first derivative of the spectrophotometric curve exhibits them. Thus, in this instance the delivery mechanism alters the cosmetic composition in such a manner prior to application to the target substrate that the desired characteristics are achieved upon delivery.

More particularly, this embodiment includes cosmetic compositions adapted for application to a substrate to provide an applied cosmetic composition having a spectrophotometric curve, wherein the applied cosmetic composition comprises at least two colorants, and wherein a first derivative of the spectrophotometric curve comprises a maximum peak in the region of from about 430 nm to about 520 nm occurs at a wavelength not greater than about 480 nm, a maximum peak in the region of from about 420 nm to about 650 nm occurs at a wavelength of from about 570 nm to about 630 nm, and a minimum valley in the region of from about 520 nm to about 580 nm has a Δ%R/Δλ of less than or equal to about 0.03, wherein R is reflectance and λ is wavelength, and wherein a first derivative of a spectrophotometric curve of each of the individual colorants does not exhibit (a), (b) and (c). In such instances, it is preferred that Δ%R/Δλ is less than or equal to about 0.015, and most preferably, less than or equal to about 0.

For example, a cosmetic composition may be packaged in a manner such that the color domains of each color are not intermixed; however, the cosmetic composition is adapted for delivery to a target substrate, i.e., skin, via a delivery mechanism, i.e. a spray device, including a mechanical sprayer and an electrostatic sprayer, an applicator pad, or the like, which affects intermixing of the color domains in a manner sufficient to provide the applied cosmetic composition with the desired spectrophotometric curve.

In a specific embodiment, the cosmetic compositions of the invention have a spectrophotometric curve whose first derivative exhibits characteristic maximum and minimum that are substantially similar to those exhibited by the first derivative of the spectral curve of skin. For example, in an exemplary embodiment of the present invention, the cosmetic compositions are provided with skin tone color and have a spectrophotometric curve with a first derivative in which a maximum peak in the region of from about 430 nm to about 520 nm occurs at a wavelength not greater than about 480 nm, a maximum peak within the region of from about 420 nm to about 650 nm that occurs at a wavelength of from about 570 nm to about 630 nm, and a minimum valley in the region of from about 520 nm to about 580 nm has a Δ%R/Δλ of less than or equal to about 0.03, wherein R is reflectance and λ is wavelength, and wherein the cosmetic composition comprises a mixture of at least two colorants, wherein a first derivative of a spectrophotometric curve of each of the individual colorants does not exhibit (a), (b) and (c). Preferably, Δ%R/Δλ is less than or equal to about 0.015, and most preferably, less than or equal to about 0.

Optional Ingredients

The compositions of the present invention may contain a variety of other components such as are conventionally used in a given product type provided that they do not unacceptably alter the benefits of the invention. These optional components should be suitable for application to mammalian skin, that is, when incorporated into the compositions they are suitable for use in contact with human skin without undue toxicity, incompatibility, instability, allergic response, and the like, within the scope of sound medical or formulator's judgment. The CTFA Cosmetic Ingredient Handbook, Second Edition (1992) describes a wide variety of nonlimiting cosmetic and pharmaceutical ingredients commonly used in the skin care industry, which are suitable for use in the compositions of the present invention. Examples of these ingredient classes include: enzymes, surfactants, abrasives, skin exfoliating agents, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents (e.g., resorcinol, sulfur, salicylic acid, erythromycin, zinc, etc.), anti-caking agents, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, polymer beads, film formers or materials, e.g., polymers, for aiding the film-forming properties and substantivity of the composition (e.g., copolymer of eicosene and vinyl pyrrolidone), humectants, opacifying agents, pH adjusters, propellants, reducing agents, sequestrants, skin bleaching agents (or lightening agents) (e.g., hydroquinone, kojic acid, ascorbic acid, magnesium ascorbyl phosphate, ascorbyl glucosamine), skin soothing and/or healing agents (e.g., panthenol and derivatives (e.g., ethyl panthenol), aloe vera, pantothenic acid and its derivatives, allantoin, bisabolol, and dipotassium glycyrrhizinate), thickeners, hydrocolloids, particular zeolites, and vitamins and derivatives thereof (e.g. tocopherol, tocopherol acetate, beta carotene, retinoic acid, retinol, retinoids, retinyl palmitate, niacin, niacinamide, and the like). The compositions of the present invention may include carrier components such as are known in the art. Such carriers can include one or more compatible liquid or solid filler diluents or vehicles that are suitable for application to skin.

The optional components useful herein can be categorized by their therapeutic or aesthetic benefit or their postulated mode of action. However, it is to be understood that the optional components useful herein can in some instances provide more than one therapeutic or aesthetic benefit or operate via more than one mode of action. Therefore, classifications herein are made for the sake of convenience and are not intended to limit the component to that particular application or applications listed. Also, when applicable, the pharmaceutically-acceptable salts of the components are useful herein.

Desquamation Actives

A safe and effective amount of a desquamation active may be added to the compositions of the present invention, preferably from about 0.1% to about 10%, more preferably from about 0.2% to about 5%, even more preferably from about 0.5% to about 4%, by weight of the composition. Desquamation actives enhance the skin appearance benefits of the present invention. For example, the desquamation actives tend to improve the texture of the skin (e.g., smoothness). One desquamation system that is suitable for use herein contains sulfhydryl compounds and zwitterionic surfactants and is described in U.S. Pat. No. 5,681,852, to Bissett, incorporated herein by reference. Another desquamation system that is suitable for use herein contains salicylic acid and zwitterionic surfactants and is described in U.S. Pat. No. 5,652,228 to Bissett, incorporated herein by reference. Zwitterionic surfactants such as described in these applications are also useful as desquamatory agents herein, with cetyl betaine being particularly preferred.

Anti-Acne Actives

The compositions of the present invention may contain a safe and effective amount of one or more anti-acne actives preferably from about 0.01% to about 50%, more preferably from about 1% to about 20%. Examples of useful anti-acne actives include resorcinol, sulfur, salicylic acid, benzoyl peroxide, erythromycin, zinc, etc. Further examples of suitable anti-acne actives are described in further detail in U.S. Pat. No. 5,607,980, issued to McAtee et al, on Mar. 4, 1997.

Anti-Wrinkle Actives/Anti-Atrophy Actives

The compositions of the present invention may contain a safe and effective amount of one or more anti-wrinkle actives or anti-atrophy actives. Exemplary anti-wrinkle/anti-atrophy actives suitable for use in the compositions of the present invention include sulfur-containing D and L amino acids and their derivatives and salts, particularly the N-acetyl derivatives, a preferred example of which is N-acetyl-L-cysteine; thiols, e.g. ethane thiol; hydroxy acids (e.g., alpha-hydroxy acids such as salicylic acid, lactic acid and glycolic acid or beta-hydroxy acids such as salicylic acid and salicylic acid derivatives such as the octanoyl derivative), phytic acid, lipoic acid; lysophosphatidic acid, and skin peel agents (e.g., phenol and the like), which enhance the keratinous tissue appearance benefits of the present invention, especially in regulating keratinous tissue condition, e.g., skin condition.

Anti-Oxidants/Radical Scavengers

The compositions of the present invention may include a safe and effective amount of an anti-oxidant/radical scavenger, preferably from about 0.1% to about 10%, more preferably from about 1% to about 5%, of the composition. The anti-oxidant/radical scavenger is especially useful for providing protection against UV radiation which can cause increased scaling or texture changes in the stratum corneum and against other environmental agents which can cause skin damage.

Anti-oxidants/radical scavengers such as ascorbic acid (vitamin C) and its salts, ascorbyl esters of fatty acids, ascorbic acid derivatives (e.g., magnesium ascorbyl phosphate, sodium ascorbyl phosphate, ascorbyl sorbate), tocopherol (vitamin E), tocopherol acetate, other esters of tocopherol, butylated hydroxy benzoic acids and their salts, 6-hydroxy-2,5,7,8-tetramethylchroman-2-carboxylic acid (commercially available under the tradename Trolox®), gallic acid and its alkyl esters, especially propyl gallate, uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, lycine pidolate, arginine pilolate, nordihydroguaiaretic acid, bioflavonoids, curcumin, lysine, methionine, proline, superoxide dismutase, silymarin, tea extracts, grape skin/seed extracts, melanin, and rosemary extracts may be used. Preferred anti-oxidants/radical scavengers are selected from tocopherol acetate, other esters of tocopherol, and mixtures thereof. Tocopherol acetate is especially preferred.

Chelators

The compositions of the present invention may contain a safe and effective amount of a chelator or chelating agent. As used herein, “chelator” or “chelating agent” means an active agent capable of removing a metal ion from a system by forming a complex so that the metal ion cannot readily participate in or catalyze chemical reactions.

A safe and effective amount of a chelating agent may be added to the compositions of the subject invention, preferably from about 0.1% to about 10%, more preferably from about 1% to about 5%, of the composition. Exemplary chelators that are useful herein are disclosed in U.S. Pat. No. 5,487,884, issued Jan. 30, 1996 to Bissett et al.; International Publication No. 91/16035, Bush et al., published Oct. 31, 1995; and International Publication No. 91/16034, Bush et al., published Oct. 31, 1995. Preferred chelators useful in compositions of the subject invention are furildioxime, furilmonoxime, and derivatives thereof.

Flavonoids

The compositions of the present invention may contain a safe and effective amount of flavonoid compound. Flavonoids are broadly disclosed in U.S. Pat. Nos. 5,686,082 and 5,686,367, both of which are herein incorporated by reference. Flavonoids suitable for use in the present invention are flavanones selected from unsubstituted flavanones, mono-substituted flavanones, and mixtures thereof; chalcones selected from unsubstituted chalcones, mono-substituted chalcones, di-substituted chalcones, tri-substituted chalcones, and mixtures thereof; flavones selected from unsubstituted flavones, mono-substituted flavones, di-substituted flavones, and mixtures thereof; one or more isoflavones; coumarins selected from unsubstituted coumarins, mono-substituted coumarins, di-substituted coumarins, and mixtures thereof; chromones selected from unsubstituted chromones, mono-substituted chromones, di-substituted chromones, and mixtures thereof; one or more dicoumarols; one or more chromanones; one or more chromanols; isomers (e.g., cis/trans isomers) thereof; and mixtures thereof. By the term “substituted” as used herein means flavonoids wherein one or more hydrogen atom of the flavonoid has been independently replaced with hydroxyl, C1-C8 alkyl, C1-C4 alkoxyl, O-glycoside, and the like or a mixture of these substituents.

Examples of suitable flavonoids include, but are not limited to, unsubstituted flavanone, mono-hydroxy flavanones (e.g., 2′-hydroxy flavanone, 6-hydroxy flavanone, 7-hydroxy flavanone, etc.), mono-alkoxy flavanones (e.g., 5-methoxy flavanone, 6-methoxy flavanone, 7-methoxy flavanone, 4′-methoxy flavanone, etc.), unsubstituted chalcone (especially unsubstituted transchalcone), mono-hydroxy chalcones (e.g., 2′-hydroxy chalcone, 4′-hydroxy chalcone, etc.), dihydroxy chalcones (e.g., 2′,4-dihydroxy chalcone, 2′,4′-dihydroxy chalcone, 2,2′-dihydroxy chalcone, 2′,3-dihydroxy chalcone, 2′,5′-dihydroxy chalcone, etc.), and tri-hydroxy chalcones (e.g., 2′,3′,4′-trihydroxy chalcone, 4,2′,4′-trihydroxy chalcone, 2,2′,4′-trihydroxy chalcone, etc.), unsubstituted flavone, 7,2′-dihydroxy flavone, 3′,4′-dihydroxy naphthoflavone, 4′-hydroxy flavone, 5,6-benzoflavone, and 7,8-benzoflavone, unsubstituted isoflavone, daidzein (7,4′-dihydroxy isoflavone), 5,7-dihydroxy-4′-methoxy isoflavone, soy isoflavones (a mixture extracted from soy), unsubstituted coumarin, 4-hydroxy coumarin, 7-hydroxy coumarin, 6-hydroxy-4-methyl coumarin, unsubstituted chromone, 3-formyl chromone, 3-formyl-6-isopropyl chromone, unsubstituted dicoumarol, unsubstituted chromanone, unsubstituted chromanol, and mixtures thereof.

Preferred for use herein are unsubstituted flavanone, methoxy flavanones, unsubstituted chalcone, 2′,4-dihydroxy chalcone, isoflavone, flavone, and mixtures thereof. More preferred are soy isoflavones.

Mixtures of the above flavonoid compounds may also be used.

The herein described flavonoid compounds are preferably present in the instant invention at concentrations of from about 0.01% to about 20%, more preferably from about 0.1% to about 10%, and still more preferably from about 0.5% to about 5%.

Anti-Inflammatory Agents

A safe and effective amount of an anti-inflammatory agent may be added to the compositions of the present invention, preferably from about 0.1% to about 10%, more preferably from about 0.5% to about 5%, of the composition.

Steroidal anti-inflammatory agents, including but not limited to, corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof may be used. The preferred steroidal anti-inflammatory for use is hydrocortisone.

A second class of anti-inflammatory agents which is useful in the compositions includes the nonsteroidal anti-inflammatory agents. The variety of compounds encompassed by this group are well-known to those skilled in the art. For detailed disclosure of the chemical structure, synthesis, side effects, etc. of non-steroidal anti-inflammatory agents, one may refer to standard texts, including Anti-inflammatory and Anti-Rheumatic Drugs, K. D. Rainsford, Vol. I-III, CRC Press, Boca Raton, (1985), and Anti-inflammatory Agents, Chemistry and Pharmacology, 1, R. A. Scherrer, et al., Academic Press, New York (1974).

Specific non-steroidal anti-inflammatory agents useful in the composition invention include, but are not limited to:

1) the oxicams, such as piroxicam, isoxicam, tenoxicam, sudoxicam, and CP-14,304;

2) the salicylates, such as aspirin, disalcid, benorylate, trilisate, safapryn, solprin, diflunisal, and fendosal;

3) the acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac;

4) the fenamates, such as mefenamic, meclofenamic, flufenamic, niflumic, and tolfenamic acids;

5) the propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; and

6) the pyrazoles, such as phenylbutazone, oxyphenbutazone, feprazone, azapropazone, and trimethazone.

Mixtures of these non-steroidal anti-inflammatory agents may also be employed, as well as the dermatologically acceptable salts and esters of these agents. For example, etofenamate, a flufenamic acid derivative, is particularly useful for topical application. Of the nonsteroidal anti-inflammatory agents, ibuprofen, naproxen, flufenamic acid, etofenamate, aspirin, mefenamic acid, meclofenamic acid, piroxicam and felbinac are preferred.

Finally, so-called “natural” anti-inflammatory agents are useful in methods of the present invention. Such agents may suitably be obtained as an extract by suitable physical and/or chemical isolation from natural sources (e.g., plants, fungi, by-products of microorganisms) or can be synthetically prepared. For example, candelilla wax, bisabolol (e.g., alpha bisabolol), aloe vera, plant sterols (e.g., phytosterol), Manjistha (extracted from plants in the genus Rubia, particularly Rubia Cordifolia), and Guggal (extracted from plants in the genus Commiphora, particularly Commiphora Mukul), kola extract, chamomile, red clover extract, and sea whip extract, may be used.

Additional anti-inflammatory agents useful herein include compounds of the Licorice (the plant genus/species Glycyrrhiza glabra) family, including glycyrrhetic acid, glycyrrhizic acid, and derivatives thereof (e.g., salts and esters). Suitable salts of the foregoing compounds include metal and ammonium salts. Suitable esters include C₂-C₂₄ saturated or unsaturated esters of the acids, preferably C₁₀-C₂₄, more preferably C₁₆-C₂₄. Specific examples of the foregoing include oil soluble licorice extract, the glycyrrhizic and glycyrrhetic acids themselves, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid, and disodium 3-succinyloxy-beta-glycyrrhetinate. Stearyl glycyrrhetinate is preferred.

Anti-Cellulite Agents

The compositions of the present invention may contain a safe and effective amount of an anti-cellulite agent. Suitable agents may include, but are not limited to, xanthine compounds (e.g., caffeine, theophylline, theobromine, and aminophylline).

Topical Anesthetics

The compositions of the present invention may contain a safe and effective amount of a topical anesthetic. Examples of topical anesthetic drugs include benzocaine, lidocaine, bupivacaine, chlorprocaine, dibucaine, etidocaine, mepivacaine, tetracaine, dyclonine, hexylcaine, procaine, cocaine, ketamine, pramoxine, phenol, and pharmaceutically acceptable salts thereof.

Tanning Actives

The compositions of the present invention may contain a safe and effective amount of a tanning active, preferably from about 0.1% to about 20% of dihydroxyacetone as an artificial tanning active.

Dihydroxyacetone, which is also known as DHA or 1,3-dihydroxy-2-propanone, is a white to off-white, crystalline powder.

Skin Lightening Agents

The compositions of the present invention may contain a skin lightening agent. When used, the compositions preferably contain from about 0.1% to about 10%, more preferably from about 0.2% to about 5%, also preferably from about 0.5% to about 2%, by weight of the composition, of a skin lightening agent. Suitable skin lightening agents include those known in the art, including kojic acid, arbutin, ascorbic acid and derivatives thereof (e.g., magnesium ascorbyl phosphate or sodium ascorbyl phosphate), and extracts (e.g., mulberry extract, placental extract). Skin lightening agents suitable for use herein also include those described in the PCT publication No. 95/34280, in the name of Hillebrand, corresponding to PCT Application No. U.S. 95/07432, filed Jun. 12, 1995; and co-pending U.S. application Ser. No. 08/390,152 filed in the names of Kvalnes, Mitchell A. DeLong, Barton J. Bradbury, Curtis B. Motley, and John D. Carter, corresponding to PCT Publication No. 95/23780, published Sep. 8, 1995.

Skin Soothing and Skin Healing Actives

A safe and effective amount of a skin soothing or skin healing active may be added to the present composition, preferably, from about 0.1% to about 30%, more preferably from about 0.5% to about 20%, still more preferably from about 0.5% to about 10%, by weight of the composition formed. Skin soothing or skin healing actives suitable for use herein include panthenoic acid derivatives (including panthenol, dexpanthenol, ethyl panthenol), aloe vera, allantoin, bisabolol, and dipotassium glycyrrhizinate.

Antimicrobial and Antifungal Actives

The compositions of the present invention may contain an antimicrobial or antifungal active. A safe and effective amount of an antimicrobial or antifungal active may be added to the present compositions, preferably, from about 0.001% to about 10%, more preferably from about 0.01% to about 5%, and still more preferably from about 0.05% to about 2%.

Examples of antimicrobial and antifungal actives include β-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, phenoxy propanol, phenoxyisopropanol, doxycycline, capreomycin, chlorhexidine, chlortetracycline, oxytetracycline, clindamycin, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, ketaconazole, amanfadine hydrochloride, amanfadine sulfate, octopirox, parachlorometa xylenol, nystatin, tolnaftate, zinc pyrithione and clotrimazole.

Preferred examples of actives useful herein include those selected from salicylic acid, benzoyl peroxide, 3-hydroxy benzoic acid, glycolic acid, lactic acid, 4-hydroxy benzoic acid, acetyl salicylic acid, 2-hydroxybutanoic acid, 2-hydroxypentanoic acid, 2-hydroxyhexanoic acid, phytic acid, N-acetyl-L-cysteine, lipoic acid, azelaic acid, arachidonic acid, benzoylperoxide, tetracycline, ibuprofen, naproxen, hydrocortisone, acetominophen, resorcinol, phenoxyethanol, phenoxypropanol, phenoxyisopropanol, 2,4,4′-trichloro-2′-hydroxy diphenyl ether, 3,4,4′-trichlorocarbanilide, octopirox, lidocaine hydrochloride, clotrimazole, miconazole, ketoconazole, neocycin sulfate, and mixtures thereof.

Sunscreen Actives

Exposure to ultraviolet light can result in excessive scaling and texture changes of the stratum corneum. Therefore, the compositions of the subject invention may contain a safe and effective amount of a sunscreen active. As used herein, “sunscreen active” includes both sunscreen agents and physical sunblocks. Suitable sunscreen actives may be organic or inorganic.

Inorganic sunscreens useful herein include the following metallic oxides; titanium dioxide having an average primary particle size of from about 15 nm to about 100 nm, zinc oxide having an average primary particle size of from about 15 nm to about 150 nm, zirconium oxide having an average primary particle size of from about 15 nm to about 150 nm, iron oxide having an average primary particle size of from about 15 nm to about 500 nm, and mixtures thereof. When used herein, the inorganic sunscreens are present in the amount of from about 0.1% to about 20%, preferably from about 0.5% to about 10%, more preferably from about 1% to about 5%, by weight of the composition.

A wide variety of conventional organic sunscreen actives are suitable for use herein. Sagarin, et al., at Chapter VIII, pages 189 et seq., of Cosmetics Science and Technology (1972), discloses numerous suitable actives. Specific suitable sunscreen actives include, for example: p-aminobenzoic acid, its salts and its derivatives (ethyl, isobutyl, glyceryl esters; p-dimethylaminobenzoic acid); anthranilates (i.e., o-amino-benzoates; methyl, menthyl, phenyl, benzyl, phenylethyl, linalyl, terpinyl, and cyclohexenyl esters); salicylates (amyl, phenyl, octyl, benzyl, menthyl, glyceryl, and di-pro-pyleneglycol esters); cinnamic acid derivatives (menthyl and benzyl esters, a-phenyl cinnamonitrile; butyl cinnamoyl pyruvate); dihydroxycinnamic acid derivatives (umbelliferone, methylumbelliferone, methylaceto-umbelliferone); trihydroxy-cinnamic acid derivatives (esculetin, methylesculetin, daphnetin, and the glucosides, esculin and daphnin); hydrocarbons (diphenylbutadiene, stilbene); dibenzalacetone and benzalacetophenone; naphtholsulfonates (sodium salts of 2-naphthol-3,6-disulfonic and of 2-naphthol-6,8-disulfonic acids); di-hydroxynaphthoic acid and its salts; o- and p-hydroxybiphenyldisulfonates; coumarin derivatives (7-hydroxy, 7-methyl, 3-phenyl); diazoles (2-acetyl-3-bromoindazole, phenyl benzoxazole, methyl naphthoxazole, various aryl benzothiazoles); quinine salts (bisulfate, sulfate, chloride, oleate, and tannate); quinoline derivatives (8-hydroxyquinoline salts, 2-phenylquinoline); hydroxy- or methoxy-substituted benzophenones; uric and violuric acids; tannic acid and its derivatives (e.g., hexaethylether); (butyl carbotol) (6-propyl piperonyl) ether; hydroquinone; benzophenones (oxybenzene, sulisobenzone, dioxybenzone, benzoresorcinol, 2,2′,4,4′-tetrahydroxybenzophenone, 2,2′-dihydroxy-4,4′-dimethoxybenzophenone, octabenzone; 4-isopropyldibenzoylmethane; butylmethoxydibenzoylmethane; etocrylene; octocrylene; [3-(4′-methylbenzylidene bornan-2-one), terephthalylidene dicamphor sulfonic acid and 4-isopropyl-dibenzoylmethane.

Of these, 2-ethylhexyl-p-methoxycinnamate (commercially available as PARSOL MCX), 4,4′-t-butyl methoxydibenzoyl-methane (commercially available as PARSOL 1789), 2-hydroxy-4-methoxybenzophenone, octyldimethyl-p-aminobenzoic acid, digalloyltrioleate, 2,2-dihydroxy-4-methoxybenzophenone, ethyl-4-(bis(hydroxy-propyl))aminobenzoate, 2-ethylhexyl-2-cyano-3,3-diphenylacrylate, 2-ethylhexyl-salicylate, glyceryl-p-aminobenzoate, 3,3,5-trimethylcyclohexylsalicylate, methylanthranilate, p-dimethyl-aminobenzoic acid or aminobenzoate, 2-ethylhexyl-p-dimethyl-amino-benzoate, 2-phenylbenzimidazole-5-sulfonic acid, 2-(p-dimethylaminophenyl)-5-sulfonicbenzoxazoic acid, octocrylene and mixtures of these compounds, are preferred.

Also particularly useful in the compositions are sunscreen actives such as those disclosed in U.S. Pat. No. 4,937,370 issued to Sabatelli on Jun. 26,1990, and U.S. Pat. No. 4,999,186 issued to Sabatelli & Spirnak on Mar. 12, 1991. The sunscreening agents disclosed therein have, in a single molecule, two distinct chromophore moieties which exhibit different ultra-violet radiation absorption spectra. One of the chromophore moieties absorbs predominantly in the UVB radiation range and the other absorbs strongly in the UVA radiation range.

Preferred members of this class of sunscreening agents are 4-N,N-(2-ethylhexyl)methyl-aminobenzoic acid ester of 2,4-dihydroxybenzophenone; N,N-di-(2-ethylhexyl)-4-aminobenzoic acid ester with 4-hydroxydibenzoylmethane; 4-N,N-(2-ethylhexyl)methyl-aminobenzoic acid ester with 4-hydroxydibenzoylmethane; 4-N,N-(2-ethylhexyl)methyl-aminobenzoic acid ester of 2-hydroxy-4-(2-hydroxyethoxy)benzophenone; 4-N,N-(2-ethylhexyl)-methylaminobenzoic acid ester of 4-(2-hydroxyethoxy)dibenzoylmethane; N,N-di-(2-ethylhexyl)-4-aminobenzoic acid ester of 2-hydroxy-4-(2-hydroxyethoxy)benzophenone; and N,N-di-(2-ethylhexyl)-4-aminobenzoic acid ester of 4-(2-hydroxyethoxy)dibenzoylmethane and mixtures thereof.

Especially preferred sunscreen actives include 4,4′-t-butylmethoxydibenzoylmethane, 2-ethylhexyl-p-methoxycinnamate, phenyl benzimidazole sulfonic acid, and octocrylene.

A safe and effective amount of the organic sunscreen active is used, typically from about 1% to about 20%, more typically from about 2% to about 10% by weight of the composition. Exact amounts will vary depending upon the sunscreen or sunscreens chosen and the desired Sun Protection Factor (SPF).

Particulate Materials

The compositions of the present invention may contain a safe and effective amount of a particulate material, preferably a metallic oxide. These particulates can be coated or uncoated, charged or uncharged. Charged particulate materials are disclosed in U.S. Pat. No. 5,997,887, to Ha, et al., incorporated herein by reference. Particulate materials useful herein include; bismuth oxychloride, iron oxide, mica, mica treated with barium sulfate and TiO2, silica, nylon, polyethylene, talc, styrene, polypropylene, ethylene/acrylic acid copolymer, titanium dioxide, iron oxide, bismuth oxychloride, sericite, aluminum oxide, silicone resin, barium sulfate, calcium carbonate, cellulose acetate, polymethyl methacrylate, and mixtures thereof.

One example of a suitable particulate material contains the material available from U.S. Cosmetics (TRONOX TiO2 series, SAT-T CR837, a rutile TiO2). Preferably, particulate materials are present in the composition in levels of from about 0.01% to about 2%, more preferably from about 0.05% to about 1.5%, still more preferably from about 0.1% to about 1%, by weight of the composition.

Conditioning Agents

The compositions of the present invention may contain a safe and effective amount of a conditioning agent selected from humectants, moisturizers, or skin conditioners. A variety of these materials can be employed and each can be present at a level of from about 0.01% to about 20%, more preferably from about 0.1% to about 10%, and still more preferably from about 0.5% to about 7% by weight of the composition. These materials include, but are not limited to, guanidine; urea; glycolic acid and glycolate salts (e.g. ammonium and quaternary alkyl ammonium); salicylic acid; lactic acid and lactate salts (e.g., ammonium and quaternary alkyl ammonium); aloe vera in any of its variety of forms (e.g., aloe vera gel); polyhydroxy alcohols such as sorbitol, mannitol, xylitol, erythritol, glycerol, hexanetriol, butanetriol, propylene glycol, butylene glycol, hexylene glycol and the like; polyethylene glycols; sugars (e.g., melibiose) and starches; sugar and starch derivatives (e.g., alkoxylated glucose, fucose); hyaluronic acid; lactamide monoethanolamine; acetamide monoethanolamine; panthenol; allantoin; and mixtures thereof. Also useful herein are the propoxylated glycerols described in U.S. Pat. No. 4,976,953, to Orr et al, issued Dec. 11, 1990.

Also useful are various C₁-C₃₀ monoesters and polyesters of sugars and related materials. These esters are derived from a sugar or polyol moiety and one or more carboxylic acid moieties.

Preferably, the conditioning agent is selected from urea, guanidine, sucrose polyester, panthenol, dexpanthenol, allantoin, glycerol, and combinations thereof.

Thickening Agents (Including Thickeners, Structuring and Gelling Agents)

The compositions of the present invention may contain a safe and effective amount of one or more thickening agents, preferably from about 0.1% to about 5%, more preferably from about 0.1% to about 4%, and still more preferably from about 0.25% to about 3%, by weight of the composition.

Classes of thickening agents include the following:

a) Carboxylic Acid Polymers

These polymers are crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol. Polymers useful in the present invention are more fully described in U.S. Pat. No. 5,087,445, to Haffey et al, issued Feb. 11, 1992; U.S. Pat. No. 4,509,949, to Huang et al, issued Apr. 5, 1985; U.S. Pat. No. 2,798,053, to Brown, issued Jul. 2, 1957; and in CTFA International Cosmetic Ingredient Dictionary, Fourth Edition, 1991, pp. 12 and 80.

Examples of commercially available carboxylic acid polymers useful herein include the carbomers, which are homopolymers of acrylic acid crosslinked with allyl ethers of sucrose or pentaerytritol. The carbomers are available as the Carbopol® 900 series from B. F. Goodrich (e.g., Carbopol® 954). In addition, other suitable carboxylic acid polymeric agents include copolymers of C₁₀₋₃₀ alkyl acrylates with one or more monomers of acrylic acid, methacrylic acid, or one of their short chain (i.e., C₁₋₄ alcohol) esters, wherein the crosslinking agent is an allyl ether of sucrose or pentaerytritol. These copolymers are known as acrylates/C₁₀₋₃₀ alkyl acrylate crosspolymers and are commercially available as Carbopol® 1342, Carbopol® 1382, Pemulen TR-1, and Pemulen TR-2, from B. F. Goodrich. Examples of carboxylic acid polymer thickeners useful herein are those selected from carbomers, acrylates/C₁₀-C₃₀ alkyl acrylate crosspolymers, and mixtures thereof.

b) Crosslinked Polyacrylate Polymers

The compositions of the present invention may contain a safe and effective amount of crosslinked polyacrylate polymers useful as thickeners or gelling agents including both cationic and nonionic polymers, with the cationics being generally preferred. Examples of useful crosslinked nonionic polyacrylate polymers and crosslinked cationic polyacrylate polymers are those described in U.S. Pat. No. 5,100,660, to Hawe et al, issued Mar. 31, 1992; U.S. Pat. No. 4,849,484, to Heard, issued Jul. 18, 1989; U.S. Pat. No. 4,835,206, to Farrar et al, issued May 30, 1989; U.S. Pat. No. 4,628,078 to Glover et al issued Dec. 9, 1986; U.S. Pat. No. 4,599,379 to Flesher et al issued Jul. 8, 1986; and EP 228,868, to Farrar et al, published Jul. 15, 1987.

c) Polyacrylamide Polymers

The compositions of the present invention may contain a safe and effective amount of polyacrylamide polymers, especially nonionic polyacrylamide polymers including substituted branched or unbranched polymers. More preferred among these polyacrylamide polymers is the nonionic polymer given the CTFA designation polyacrylamide and isoparaffin and laureth-7, available under the Tradename Sepigel 305 from Seppic Corporation (Fairfield, N.J.).

Other polyacrylamide polymers useful herein include multi-block copolymers of acrylamides and substituted acrylamides with acrylic acids and substituted acrylic acids. Commercially available examples of these multi-block copolymers include Hypan SR150H, SS500V, SS500W, SSSA100H, from Lipo Chemicals, Inc., (Patterson, N.J.).

d) Polysaccharides

A wide variety of polysaccharides are useful herein. “Polysaccharides” refer to gelling agents which contain a backbone of repeating sugar (i.e., carbohydrate) units. Examples of polysaccharide gelling agents include those selected from cellulose, carboxymethyl hydroxyethylcellulose, cellulose acetate propionate carboxylate, hydroxyethylcellulose, hydroxyethyl ethylcellulose, hydroxypropylcellulose; hydroxypropyl methylcellulose, methyl hydroxyethylcellulose, microcrystalline cellulose, sodium cellulose sulfate, and mixtures thereof. Also useful herein are the alkyl substituted celluloses. In these polymers, the hydroxy groups of the cellulose polymer is hydroxyalkylated (preferably hydroxyethylated or hydroxypropylated) to form a hydroxyalkylated cellulose which is then further modified with a C₁₀-C₃₀ straight chain or branched chain alkyl group through an ether linkage. Typically these polymers are ethers of C₁₀-C₃₀ straight or branched chain alcohols with hydroxyalkylcelluloses. Examples of alkyl groups useful herein include those selected from stearyl, isostearyl, lauryl, myristyl, cetyl, isocetyl, cocoyl (i.e. alkyl groups derived from the alcohols of coconut oil), palmityl, oleyl, linoleyl, linolenyl, ricinoleyl, behenyl, and mixtures thereof. Preferred among the alkyl hydroxyalkyl cellulose ethers is the material given the CTFA designation cetyl hydroxyethylcellulose, which is the ether of cetyl alcohol and hydroxyethylcellulose. This material is sold under the tradename Natrosol® CS Plus from Aqualon Corporation (Wilmington, Del.).

Other useful polysaccharides include scleroglucans which are a linear chain of (1-3) linked glucose units with a (1-6) linked glucose every three units, a commercially available example of which is Clearogel™ CS11 from Michel Mercier Products Inc. (Mountainside, N.J.).

e) Gums

Other thickening and gelling agents useful herein include materials which are primarily derived from natural sources. Examples of these gelling agent gums include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, nafto gum, potassium alginate, potassium carrageenan, propylene glycol alginate, sclerotium gum, sodium carboyxmethyl dextran, sodium carrageenan, tragacanth gum, xanthan gum, and mixtures thereof.

f) Additional Thickening Agents

Suitable thickening agents can be selected from the group consisting of silicones, waxes, clays, silicas, salts, natural and synthetic esters, fatty alcohols, and mixtures thereof. Nonlimiting examples of these structuring or thickening agents are described below.

Suitable silicones include alkyl siloxane gellants, high molecular weight dimethicones (fluids greater than 1000 mPas), and high molecular weight alkyl, hydroxyl, carboxyl, amino, and/or fluoro-substituted dimethicones (fluids greater than 1000 mPas). Preferred silicone gellants are described in U.S. Pat. Nos. 5,654,362 and 5,880,210, and include cyclomethicone and dimethicone crosspolymers (e.g., Dow Corning 9040).

Waxes can be defined as lower-melting organic mixtures or compounds of high molecular weight, solid at room temperature and generally similar in composition to fats and oils except that they contain no glycerides. Some are hydrocarbons, others are esters of fatty acids and alcohols. Suitable waxes may be selected from the group consisting of natural waxes including animal waxes, vegetable waxes, and mineral waxes, and synthetic waxes including petroleum waxes, ethylenic polymers, hydrocarbon waxes (e.g., Fischer-Tropsch waxes), ester waxes, silicone waxes, and mixtures thereof. Synthetic waxes include those disclosed in Warth, Chemistry and Technology of Waxes, Part 2, Reinhold Publishing (1956); herein incorporated by reference.

Specific examples of waxes include beeswax, lanolin wax, shellac wax, carnauba, candelilla, bayberry, jojoba esters, behenic acid waxes (e.g., glyceryl behenate which is available from Gattifosse as Compritol®), ozokerite, ceresin, paraffin, microcrystalline waxes, polyethylene homopolymers, polymers comprising ethylene oxide or ethylene (e.g., long chained polymers of ethylene oxide combined with a dihydric alcohol, namely polyoxyethylene glycol, such as Carbowax available from Carbide and Carbon Chemicals company; long-chained polymers of ethylene with OH or another stop length grouping at end of chain, including Fischer-Tropsch waxes as disclosed in Warth, supra, at pages 465-469 and specifically including Rosswax available from Ross Company and PT-0602 available from Astor Wax Company), C₂₄₋₄₅ alkyl methicones, C₈ to C₅₀ hydrocarbon waxes, alkylated polyvinyl pyrrolidones (e.g., “Ganex” alkylated polyvinylpyrrolidines available from the ISP Company), fatty alcohols from C20 to C60 (e.g., “Unilins”, available from Petrolite Corporation), and mixtures thereof.

Water dispersible and oil dispersible clays may be useful to provide structure or thickening. Suitable clays can be selected, e.g., from montmorillonites, bentonites, hectorites, attapulgites, sepiolites, laponites, silicates and mixtures thereof.

Suitable water dispersible clays include bentonite and hectorite (such as Bentone EW, LT from Rheox); magnesium aluminum silicate (such as Veegum from Vanderbilt Co.); attapulgite (such as Attasorb or Pharamasorb from Engelhard, Inc.); laponite and montmorillonite (such as Gelwhite from ECC America); and mixtures thereof.

Suitable oil dispersible clays include organophilically modified bentonites, hectorites and attapulgites. Specific commercially available examples of these clays include Bentone 34 (Rheox Corp.)—Quaternium-18 Bentonite; Tixogel VP (United Catalysts)—Quaternium-18 Bentonite; Bentone 38 (Rheox Corp.)—Quaternium-18 Hectorite; Bentone SD-3 (Rheox Corp.)—Dihydrogenated Tallow Benzylmonium Hectorite; Bentone 27 (Rheox Corp.)—Stearalkonium Hectorite; Tixogel LG (United Catalysts)—Stearalkonium Bentonite; Claytone 34 (Southern Clay) Quaternium-18 Bentonite; Claytone 40 (Southern Clay) Quaternium-18 Bentonite; Claytone AF (Southern Clay) Stearalkonium Bentonite; Claytone APA (Southern Clay) Stearalkonium Bentonite; Claytone GR (Southern Clay) Quaternium-18/Benzalkonium Bentonite; Claytone HT (Southern Clay) Quaternium-18/Benzalkonium Bentonite; Claytone PS (Southern Clay) Quaternium-18/Benzalkonium Bentonite; Claytone XL (Southern Clay) Quaternium-18 Bentonite; and Vistrol 1265 (Cimibar)—Organophilic Attapulgite. These organophilic clays can be purchased as pre-dispersed organophilic clay in either an oil or an organic solvent. The materials are in the form of a heavy paste that can be readily dispersed into the formulation. Such materials include Mastergels by Rheox, United Catalysts, and Southern Clay.

Other thickening agents include fumed silicas and alkali metal or ammonium halides. Examples of fumed silicas include Aerosil 200, Aerosil 300, and the alkyl-substituted fumed silicas such as Aerosil R-100, 200, 800, and 900 series of materials, all available from the DeGussa Corporation.

Preferred thickening agents are those that are substantially inert to the distribution of charge through a fluid, e.g., waxes and high molecular weight silicones and hydrocarbons.

Additional Powdered Ingredients

Suitable powders include various organic and inorganic pigments that color the composition or skin. Organic pigments are generally various types including azo, indigoid, triphenylmethane, anthraquinone, and xanthine dyes which are designated as D&C and FD&C blues, browns, greens, oranges, reds, yellows, etc. Inorganic pigments are generally insoluble metallic salts of certified color additives, referred to as lakes or iron oxides. Suitable pigments include those generally recognized as safe, and listed in C.T.F.A. Cosmetic Ingredient Handbook, First Edition, Washington D.C. (1988), incorporated herein by reference. Specific examples are red iron oxide, yellow iron oxide, black iron oxide, brown iron oxide, ultramarine, FD&C Red, Nos. 2, 5, 6, 7, 10, 11, 12, 13, 30 and 34; FD&C Yellow No. 5, Red 3, 21, 27, 28, and 33 Aluminum Lakes, Yellow 5, 6, and 10 Aluminum Lakes, Orange 5 Aluminum Lake, Blue 1 Aluminum Lake, Red 6 Barium Lake, Red 7 Calcium Lake, and the like.

Other useful powder materials include talc, mica, titanated mica (mica coated with titanium dioxide), iron oxide titanated mica, magnesium carbonate, calcium carbonate, magnesium silicate, silica (including spherical silica, hydrated silica and silica beads), titanium dioxide, zinc oxide, nylon powder, polyethylene powder, ethylene acrylates copolymer powder, methacrylate powder, polystyrene powder, silk powder, crystalline cellulose, starch, bismuth oxychloride, guanine, kaolin, chalk, diatomaceous earth, microsponges, boron nitride and the like. Additional powders useful herein are described in U.S. Pat. No. 5,505,937 issued to Castrogiovanni et al. Apr. 9, 1996.

Of the components useful as a matte finishing agents, low luster pigment, talc, polyethylene, hydrated silica, kaolin, titanium dioxide, titanated mica and mixtures thereof are preferred.

Micas, boron nitride and ethylene acrylates copolymer (e.g., EA-209 from Kobo) are preferred for imparting optical blurring effects through light diffraction and for improving skin feel, e.g., by providing a lubricious feel. Another particulate material for improving skin feel is SPCAT 12 (a mixture of talc, polyvinylidene copolymer, and Isopropyl titanium triisostearate).

Preferred powders for absorbing oil are spherical, nonporous particles, more preferably having a particle size less than 25 microns. Examples of some preferred oil absorbing powders are Coslin C-100 (a spherical oil absorber commercially available from Englehard), Tospearl (spherical silica commercially available Kobo Industries), ethylene acrylates copolymer such as noted above, and SPCAT 12.

The powders may be surface treated with one or more agents, e.g., with lecithin, amino acids, mineral oil, silicone oil, or various other agents, which coat the powder surface, for example, to render the particles hydrophobic or hydrophilic. Such treatment may be preferred to improve ease of formulation and stability.

Materials for Enhancing Wear or Transfer Resistance

One or more materials for imparting wear and/or transfer resistant properties, e.g., via film forming or substantive properties, may be used in the present compositions. Such materials are typically used in an amount of from about 0.5% to about 20%.

Such materials include film forming polymeric materials. While the level of film forming polymeric material may vary, typically the film forming polymeric material is present in levels of from about 0.5% to about 20% (e.g., from about 1 to about 15%), preferably from about 0.5% to about 10% by weight, more preferably from about 1% to about 8% by weight. Preferred polymers form a non-tacky film which is removable with water used with cleansers such as soap.

Examples of suitable film forming polymeric materials include:

a) sulfopolyester resins, such as AQ sulfopolyester resins, such as AQ29D, AQ35S, AQ38D, AQ38S, AQ48S, and AQ55S (available from Eastman Chemicals);

b) polyvinylacetate/polyvinyl alcohol polymers, such as Vinex resins available from Air Products, including Vinex 2034, Vinex 2144, and Vinex 2019;

c) acrylic resins, including water dispersible acrylic resins available from National Starch under the trade name “Dermacryl”, including Dermacryl LT;

d) polyvinylpyrrolidones (PVP), including Luviskol K17, K30 and K90 (available from BASF), water soluble copolymers of PVP, including PVP/VA S-630 and W-735 and PVP/dimethylaminoethylmethacrylate Copolymers such as Copolymer 845 and Copolymer 937 available from ISP, as well as other PVP polymers disclosed by E. S. Barabas in the Encyclopedia of Polymer Science and Engineering, 2 Ed., Vol. 17, pp. 198-257;

e) high molecular weight silicones such as dimethicone and organic-substituted dimethicones, especially those with viscosities of greater than about 50,000 mPas;

f) high molecular weight hydrocarbon polymers with viscosities of greater than about 50,000 mpas;

g) organosiloxanes, including organosiloxane resins, fluid diorganopolysiloxane polymers and silicone ester waxes.

Examples of these polymers and cosmetic compositions containing them are found in PCT publication Nos. WO96/33689, published Oct. 31, 1996; WO97/17058, published May 15, 1997; and U.S. Pat. No. 5,505,937 issued to Castrogiovanni et al. Apr. 9, 1996, all incorporated herein by reference. Additional film forming polymers suitable for use herein include the water-insoluble polymer materials in aqueous emulsion and water soluble film forming polymers described in PCT publication No. WO98/18431, published May 7, 1998, incorporated herein by reference. Examples of high molecular weight hydrocarbon polymers with viscosities of greater than about 50,000 mPas include polybutene, polybutene terephthalate, polydecene, polycyclopentadiene, and similar linear and branched high molecular weight hydrocarbons.

Preferred film forming polymers include organosiloxane resins comprising combinations of R₃SiO_{½} “M” units, R₂SiO “D” units, RSiO_{3/2} “T” units, SiO₂ “Q” units in ratios to each other that satisfy the relationship RₙSiO_{(4-n)/2} where n is a value between 1.0 and 1.50 and R is a methyl group. Note that a small amount, up to 5%, of silanol or alkoxy functionality may also be present in the resin structure as a result of processing. The organosiloxane resins must be solid at about 25° C. and have a molecular weight range of from about 1,000 to about 10,000 grams/mole. The resin is soluble in organic solvents such as toluene, xylene, isoparaffins, and cyclosiloxanes or the volatile carrier, indicating that the resin is not sufficiently crosslinked such that the resin is insoluble in the volatile carrier. Particularly preferred are resins comprising repeating monofunctional or R₃SiO_{½} “M” units and the quadrafunctional or SiO₂ “Q” units, otherwise known as “MQ” resins as disclosed in U.S. Pat. No. 5,330,747, Krzysik, issued Jul. 19, 1994, incorporated herein by reference. In the present invention the ratio of the “M” to “Q” functional units is preferably about 0.7 and the value of n is 1.2. Organosiloxane resins such as these are commercially available such as Wacker 803 and 804 available from Wacker Silicones Corporation of Adrian Michigan, and G. E. 1170-002 from the General Electric Company.

Other materials for enhancing wear or transfer resistance include trimethylated silica. Suitable silicas of this type and cosmetic compositions containing them are described in U.S. Pat. No. 5,800,816 issued to Brieva et al., incorporated herein by reference.

Emulsifiers

The compositions hereof may contain one or more emulsifiers, e.g., to enhance the formation and stability of the composition. Compositions of the invention typically comprise from about 0.5% to about 10%, preferably from about 1% to about 5%, more preferably from about 1.5% to about 3% of one or more emulsifiers.

The hydrophilic-lipophilic balance value of the emulsifier (herein referred to as HLB) is chosen so as to optimally lower the interfacial tension between two phases of significantly different surface tension. For a polar-in-non-polar system, HLB ranges are typically from about 4 to about 8. For a non-polar-in-polar system, HLB ranges are typically from about 12 to about 20. HLB factors are described in Wilkinson and Moore, Harry's Cosmeticology, 7th Ed. 1982, p. 738. and Schick and Fowkes, Surfactant Science Series, Vol. 2, Solvent Properties of Surfactant Solutions, p 607, incorporated herein by reference. Exemplary emulsifiers include those disclosed in the C.T.F.A. Cosmetic Ingredient Handbook, 3rd Ed., Cosmetic and Fragrance Assn., Inc., Washington D.C. (1982) pp. 587-592; and Remington's Pharmaceutical Sciences, 15th Ed. 1975, pp. 335-337; and McCutcheon's Volume 1, Emulsifiers & Detergents, 1994, North American Edition, pp. 236-239; all herein incorporated by reference.

Particularly useful emulsifiers for the present compositions include polydiorganosiloxane-polyoxyalkylene copolymers. Such polymers are described in U.S. Pat. No. 4,268,499, incorporated herein by reference. Suitable copolymers of this type are known and many are available commercially. A preferred emulsifier of this type is known by its CTFA designation as dimethicone copolyol. Preferred emulsifiers are further disclosed in U.S. Pat. No. 5,143,722, incorporated herein by reference.

Another preferred class of emulsifiers are high molecular weight polymeric emulsifiers such as are effective for stabilizing glycol/polyol-in-hydrocarbon systems (e.g., Arlacel P135 commercially available from Unichema).

Co-solubilizers

The compositions hereof may contain one or more co-solubilizers, e.g., to enhance the formation and stability of the composition. The co-solubilizer is especially useful to bridge compatibility of two materials which are normally incompatible, resulting in the creation of a single, stable phase. Co-solubilizers may therefore be particularly preferred in the single phase electrostatically sprayable compositions described herein. When used, compositions of the invention typically comprise from about 0.5% to about 10%, preferably from about 1% to about 5%, more preferably from about 1.5% to about 3% co-solubilizer.

Suitable co-solubilizers are best chosen using a solubility parameter scale as is described in “Solubility: Effects in Product, Package, Penetration, and Preservation,” by C. D. Vaughan, Cosmetics and Toiletries, Vol. 103, October 1988. Based on the solubility parameter of two incompatible materials, a third material with a solubility parameter in between that of the two incompatible materials may sometimes be found which is independently compatible with the two incompatible materials. When all three materials are then combined, they may exhibit the properties of a single stable phase, as could be measured, visually for example, via a light microscope.

Co-solubilizers can be polar fluids, non-polar fluids, polar aprotic solvents, or amphiphilic materials and are chosen from these broad categories to fit the needs of the two incompatible materials to create a single phase.

Particularly useful co-solubilizers include the polydiorganosiloxane-polyoxyalkylene copolymers described, including the polymers described in U.S. Pat. No. 4,268,499, as well as the surfactants disclosed in U.S. Pat. No. 5,143,722. Dimethicone copolyol is preferred.

Composition Preparation

The compositions useful for the methods of the present invention are generally prepared by conventional methods such as are known in the art of making topical compositions. Such methods typically involve mixing of the ingredients in one or more steps to a relatively uniform state, with or without heating, cooling, application of vacuum, and the like.

Methods for Providing Cosmetic Compositions and Associated Methods of Use

The present invention also relates to various methods for providing cosmetic compositions having a desired spectral curve exhibiting maximum and minimum first derivative characteristics. In one embodiment, a cosmetic composition with a skin tone color is provided by a method that comprises adding at least two colorants to the cosmetic composition. In a specific embodiment, the step of adding at least two colorants may comprise delivering to the cosmetic composition at least two discrete color domains as described above. In another specific embodiment, the step of adding at least two colorants comprises physically admixing at least two different colorants with the cosmetic composition. Colorants suitable for such physical mixing are those described above.

In another embodiment, the invention is directed to methods for providing a colorant mixture to spectrophotometrically match a cosmetic composition to a target substrate like skin. Methods according to this embodiment comprise: a) determining a mixture of colorants adapted to provide a cosmetic composition having a spectrophotometric curve, wherein a first derivative of the spectrophotometric curvecomprises: i) a maximum peak in the region of from about 430 nm to about 520 nm occurs at a wavelength not greater than about 480 nm; ii) a maximum peak in the region of from about 420 nm to about 650 nm occurs at a wavelength of from about 570 nm to about 630 nm; and iii) a minimum valley in the region of from about 520 nm to about 580 nm has a Δ%R/Δλ of less than or equal to about 0.03, wherein R is reflectance and λ is wavelength; and b) admixing the colorants to form a mixture; wherein a first derivative of a spectrophotometric curve of each of the individual colorants does not exhibit (a), (b) and (c). In a specific embodiment, the method may further comprise the step of adding the mixture to the cosmetic composition. As used herein, “target substrate” according to these methods may include, but is not limited to, mammalian skin, particularly human skin.

In a further embodiment, the invention includes methods for providing discrete color domains for spectrophotometrically matching a cosmetic composition to a target substrate. These methods comprise the steps of: a) determining a combination of colorants which form discrete color domains adapted to provide a cosmetic composition having a spectrophotometric curve, wherein a first derivative of the spectrophotometric curve comprises: i) a maximum peak in the region of from about 430 nm to about 520 nm occurs at a wavelength not greater than about 480 nm; ii) a maximum peak in the region of from about 420 nm to about 650 nm occurs at a wavelength of from about 570 nm to about 630 nm; and iii) a minimum valley in the region of from about 520 nm to about 580 nm has a Δ%R/Δλ of less than or equal to about 0.03, wherein R is reflectance and λ is wavelength; and b) depositing the colorants into at least two discrete color domains; wherein a first derivative of a spectrophotometric curve of each of the individual colorants does not exhibit (a), (b) and (c). In a specific embodiment, methods may further comprise the step of adding the color domains to the cosmetic composition. In each of the aforementioned methods as well as those that follow, it is preferred that Δ%R/Δλ is less than or equal to about 0.015, and most preferred that it is less than or equal to about 0.

Applicants have additionally found that the compositions of the present invention are useful in a variety of applications directed to enhancing the appearance of target substrates like skin, oral mucosa, hair, nails, and cuticles. The methods of use for the compositions disclosed and claimed herein include, but are not limited to: 1) methods of improving the natural appearance of skin; 2) methods of applying a color cosmetic to skin; 3) methods of providing UV protection to skin; 4) methods of masking the appearance of cellulite; 5) methods of preventing, retarding, and/or controlling the appearance of oil; 6) methods of modifying the feel and texture of skin; 7) methods of providing even skin tone; 8) methods of masking the appearance of spider vessels and varicose veins; 9) methods of masking the appearance of vellus hair on skin; 10) methods of concealing blemishes and/or imperfections in human skin, including pimples, acne, age spots, freckles, moles, scars, under eye circles, birth marks, post-inflammatory hyperpigmentation, etc.; 11) methods of enhancing or modifying skin color such as lightening, darkening, making more pink, making more yellow, making less dull, making less ashy, making less orange, making more radiant; 12) methods of artificial tanning; 13) methods of concealing vitiligo; 4) methods of concealing damage incurred to the skin as a result of trauma, e.g., cosmetic surgery, burns, stretching of skin, etc.; and 15) methods of concealing wrinkles, fine lines, pores, uneven skin surfaces, etc. Each of the methods discussed herein involve topical application of the claimed compositions to the target substrate, particularly skin.

### EXAMPLES

Advantages and improvements of the compositions and methods of the present invention are demonstrated in the following examples. The examples are illustrative only and are not intended to limit or preclude other embodiments of the invention.

### Example 1

In this example, the Kubelka-Munk equation is used to determine a physical mixture of colorants to provide a cosmetic composition with a desired spectrophotometric curve. The example was prepared by adding the first 8 ingredients in a vessel and agitating. The remaining 2 ingredients were slurried together prior to addition and then added to the main mixture and mixed.

| Weight % | Material |
|---|---|
| 83.44 | Purified Water |
| 8.03 | Styrene/Acrylates Copolymer and Dioctyl Sodium |
| | Sulfoccuinate and Phenoxyethanol and Methylparaben |
| | and Butylparaben and Ethylparaben and Propylparaben |
| | and Titanium Dioxide (nominally 70% of this material) |
| 0.046 | FD&C Yellow No. 5 |
| 0.047 | D&C Yellow No. 10 |
| 0.0013 | D&C Yellow No. 8 |
| 0.0046 | D&C Green No. 5 |
| 0.0045 | D&C Red 33 |
| 2.41 | Carmine (nominally 5.6% of this material) and Mono |
| | Propylene Glycol and Glucose Syrup |
| 0.83 | Xanthan Gum |
| 5.18 | Glycerine |

### Example 2

A cosmetic composition is made by combining the following, materials in a vessel.

| Materials | % |
|---|---|
| Silicone treated Titanium dioxide (rutile) | 8.25 |
| Propyl Paraben | 0.10 |
| Ethyl Paraben | 0.19 |
| Cyclomethicone (&) Dimethicone crosspolymer | 50.88 |
| Dimethicone copolyol crosspolymer | 2.48 |
| Cyclopentasiloxane (&) Dimethicone copolyol | 2.39 |
| Benzyl Alcohol | 0.24 |
| D&C Green 5 encapsulate nominally 2.5% dye | 0.16 |
| FD&C Yellow 5 encapsulate nominally 8.5% dye | 0.76 |
| D&C Yellow 10 encapsulate nominally 10% dye | 0.80 |
| D&C Red 33 encapsulate nominally 2.5% dye | 0.01 |
| Carmine encapsulate nominally 12.5% dye | 1.13 |
| Cyclopentasiloxane (D5) | q.s. |

### Example 3

A cosmetic composition is made by combining the following materials in a vessel

| Materials | % |
|---|---|
| Silicone treated Titanium dioxide (rutile) | 8.25 |
| Propyl Paraben | 0.1 |
| Ethyl Paraben | 0.19 |
| Cyclomethicone (&) Dimethicone crosspolymer | 38 |
| Dimethicone copolyol crosspolymer | 2.5 |
| Cyclopentasiloxane (&) Dimethicone copolyol | 2.5 |
| Water for Irrigation USP | 7.5 |
| Glycerin | 10 |
| Methyl Paraben | 0.1 |
| Disodium EDTA | 0.1 |
| Benzyl Alcohol | 0.24 |
| D&C Green 5 encapsulate nominally 2.5% dye | 0.16 |
| FD&C Yellow 5 encapsulate nominally 8.5% dye | 0.76 |
| D&C Yellow 10 encapsulate nominally 10% dye | 0.80 |
| D&C Red 33 encapsulate nominally 2.5% dye | 0.013 |
| Carmine encapsulate nominally 12.5% dye | 1.13 |
| Cyclopentasiloxane (D5) | q.s. |

### Example 4

A cosmetic composition is made by combining the following materials in a vessel

| Materials | % |
|---|---|
| Silicone treated Titanium dioxide (rutile) | 8.25 |
| Propyl Paraben | 0.10 |
| Ethyl Paraben | 0.19 |
| Cyclomethicone (&) Dimethicone crosspolymer | 35-55 |
| Dimethicone copolyol crosspolymer | 2.48 |
| Cyclopentasiloxane (&) Dimethicone copolyol | 2.39 |
| Benzyl Alcohol | 0.24 |
| D&C Green 5 encapsulate nominally 2.5% dye | 0-2 |
| FD&C Yellow 5 encapsulate nominally 8.5% dye | 0.02-3.80 |
| D&C Yellow 10 encapsulate nominally 10% dye | 0.02-4.00 |
| D&C Yellow 8 encapsulate nominally 2.5% dye | 0-0.5 |
| D&C Red 33 encapsulate nominally 2.5% dye | 0-0.5 |
| Carmine encapsulate nominally 12.5% dye | 0.02-5.75 |
| Optional ingredients | 0-15 |
| Cyclopentasiloxane (D5) | q.s. |

Colorants are combined in ratios that result in the correct curve shape and nominal Lch desired.

### Example 5

A cosmetic composition is made by combining the following materials in a vessel

| Materials | % |
|---|---|
| Silicone treated Titanium dioxide | 4-15 |
| Propyl Paraben | 0.10 |
| Ethyl Paraben | 0.19 |
| Cyclomethicone (&) Dimethicone crosspolymer | 35-55 |
| Dimethicone copolyol crosspolymer | 2.48 |
| Cyclopentasiloxane (&) Dimethicone copolyol | 2.39 |
| Benzyl Alcohol | 0.24 |
| D&C Green 5 encapsulate nominally 2.5% dye | 0-4 |
| FD&C Yellow 5 encapsulate nominally 8.5% dye | 0.01-7 |
| D&C Yellow 10 encapsulate nominally 10% dye | 0.01-7.5 |
| D&C Yellow 8 encapsulate nominally 2.5% dye | 0-1 |
| D&C Red 33 encapsulate nominally 2.5% dye | 0-1 |
| Carmine encapsulate nominally 12.5% dye | 0.01-10.5 |
| Optional ingredients | 0-15 |
| Cyclopentasiloxane (D5) | q.s. |

Colorants are combined in ratios that result in the correct curve shape and nominal Lch desired.

### Example 6 & 7

A cosmetic composition is made by combining the following materials in a vessel

| | Example 6 | Example 7 |
|---|---|---|
| Materials | % | % |
| Silicone treated Titanium dioxide | 1 | 1 |
| Propyl Paraben | 0.1 | 0.1 |
| Ethyl Paraben | 0.19 | 0.19 |
| Cyclomethicone (&) Dimethicone crosspolymer | 38 | 38 |
| Dimethicone copolyol crosspolymer | 2.5 | 2.5 |
| Cyclopentasiloxane (&) Dimethicone copolyol | 2.5 | 2.5 |
| Water for Irrigation USP | 7.5 | 7.5 |
| Glycerin | 10 | 10 |
| Methyl Paraben | 0.1 | 0.1 |
| Disodium EDTA | 0.1 | 0.1 |
| Benzyl Alcohol | 0.24 | 0.24 |
| D&C Green 5 | 0.0004 | 0.0001 |
| FD&C Yellow 5 | 0.004 | 0.001 |
| D&C Yellow 10 | 0.004 | 0.001 |
| D&C Red 33 | 0.0003 | 0.0001 |
| Carmine | 0.02 | 0.005 |
| Cyclopentasiloxane (D5) | q.s. | q.s |

### Example 8

A cosmetic composition is made by combining the following materials in a vessel

| Materials | % |
|---|---|
| Silicone treated Titanium dioxide | 1 |
| Propyl Paraben | 0.1 |
| Ethyl Paraben | 0.19 |
| Cyclomethicone (&) Dimethicone crosspolymer | 38 |
| Dimethicone copolyol crosspolymer | 25 |
| Cyclopentasiloxane (&) Dimethicone copolyol | 2.5 |
| Water for Irrigation USP | 7.5 |
| Glycerin | 10 |
| Methyl Paraben | 0.1 |
| Disodium EDTA | 0.1 |
| Benzyl Alcohol | 0.24 |
| D&C Green 5 encapsulate nominally 2.5% dye | 0.019 |
| FD&C Yellow 5 encapsulate nominally 8.5% dye | 0.092 |
| D&C Yellow 10 encapsulate nominally 10% dye | 0.097 |
| D&C Red 33 encapsulate nominally 2.5% dye | 0.0016 |
| Carmine encapsulate nominally 12.5% dye | 0.14 |
| Cyclopentasiloxane (D5) | q.s. |

### Example 9

A cosmetic composition is made by combining the following materials in a vessel.

| Materials | % |
|---|---|
| Silicone treated Titanium dioxide | 0.1-4% |
| Propyl Paraben | 0.10 |
| Ethyl Paraben | 0.19 |
| Cyclomethicone (&) Dimethicone crosspolymer | 35-55 |
| Dimethicone copolyol crosspolymer | 2.48 |
| Cyclopentasiloxane (&) Dimethicone copolyol | 2.39 |
| Benzyl Alcohol | 024 |
| D&C Green 5 encapsulate nominally 2.5% dye | 0-1 |
| FD&C Yellow 5 encapsulate nominally 8.5% dye | 0.0002-2 |
| D&C Yellow 10 encapsulate nominally 10% dye | 0.0002-2 |
| D&C Yellow 8 encapsulate nominally 2.5% dye | 0-0.25 |
| D&C Red 33 encapsulate nominally 2.5% dye | 0-0.25 |
| Carmine encapsulate nominally 12.5% dye | 0.0002-3 |
| Optional ingredients | 0-15 |
| Cyclopentasiloxane (D5) | q.s. |

Colorants are combined in ratios that result in the correct curve shape and nominal Lch desired.

### Example 10-11

A color cosmetic composition in the form of a lipstick product is made by combining the ingredients as detailed below.

| | Example 10 | Example 11 |
|---|---|---|
| Ingredients | Wt % | Wt % |
| MQ resin | 20.84 | 20.84 |
| Dimethicone gum | 14.03 | 14.03 |
| Hectorite clay | 3.09 | 3.09 |
| Propylene Carbonate | 0.93 | 0.93 |
| Isododecane | 52.42 | 52.36 |
| TiO2 (STNP -CR837) | 2.04 | 2.04 |
| Encapsulated D&C Green 5 dye | 0.01 | 0.01 |
| Encapsulated FD&C Yellow 5 dye | 0.11 | 0.09 |
| Encapsulated D&C Yellow 10 dye | 0.14 | 0.15 |
| Encapsulated Carminic acid dye | 0.42 | 0.45 |
| Encapsulent shell | 5.97 | 6.01 |
| Total | 100 | 100 |

Dissolve the MQ resin and Dimethicone gum into the appropriate amount of isododecane solvent. Mill the isododecane, hectorite clay and propylene carbonate into a paste. Combine the paste, resin and gum mixture and then mill. Add the TiO2 to the above mixture and mill until uniform. Add the encapsulated dyes to the above TiO2 monochromatic mixture and mill.

### Example 12

A colorant mixture is prepared by mixing the following ingredients together in a vessel. The colorant mixture can then be used in lieu of other colorants into any of the exemplary compositions 1-11.

| Colorant | Wt % |
|---|---|
| FD&C Yellow 5 | 0.40 |
| D&C Yellow 10 | 0.56 |
| D&C Red 36 | 1.70 |
| Red Iron Oxide | 0.71 |
| D&C Red 7 Ca Lake | 0.38 |
| 68% TiO₂ | 96.25 |

### Example 13

A cosmetic composition is made as directed below.

| Part | Ingredient | Wt % |
|---|---|---|
| B | Cyclomethicone & Dimethicone Crosspolymer | 64.00 |
| A | Cyclomethicone | 14.88 |
| A | Cyclopentasiloxane & Dimethicone Copolyol | 0.32 |
| A | Trimethylsiloxysilicate | 0.80 |
| | Colored Encapsulates within particle size 30-75 um: | |
| C | Chromium Hydroxide Green | 0.30 |
| C | TiO2 (anatase) | 13.95 |
| C | TiO2 (rutile) | 1.77 |
| C | D&C Red 27 Al Lake | 0.28 |
| C | D&C Yellow 10 Al Lake | 2.51 |
| C | D&C Red 7 Ca Lake | 1.19 |

First, weigh and add Part A ingredients into glass jar; mix until mixture is clear. Add Part B ingredients and mix with spatula. Add Part C ingredients and mix mixture with spatula.

### Examples 14-16

A colorant mixture is prepared by mixing the following ingredients together in a vessel. The colorant mixture can then be used in lieu of other colorants into exemplary composition 13.

| Colored Encapsulates within | Example 14 | Example 15 | Example 16 |
|---|---|---|---|
| particle size 30-75 um | (%) | (%) | (%) |
| Chromium Hydroxide Green | 17.93 | 13.12 | 12.49 |
| D&C Yellow 10 | 46.79 | 42.36 | 39.45 |
| D&C Red 7 Ca Lake | 21.03 | 19.26 | 14.08 |
| D&C Red 27 Al Lake | 6.86 | 7.66 | 7.13 |
| 68% TiO₂ | 7.38 | 17.6 | 26.85 |

The specific embodiments and examples set forth above are provided for illustrative purposes only and are not intended to limit the scope of the following claims. Additional embodiments of the invention and advantages provided thereby will be apparent to one of ordinary skill in the art and are within the scope of the claims.

What is claimed is:

1. A cosmetic composition having a spectrophotometric curve, wherein a first derivative of the spectrophotometric curve comprises: a) a maximum peak in the region of from about 430 nm to about 520 nm occurs at a wavelength not greater than about 480 nm; b) a maximum peak in the region of from about 420 nm to about 650 nm occurs at a wavelength of from about 570 nm to about 630 nm; and c) a minimum valley in the region of from about 520 nm to about 580 nm has Δ%R/Δλ of less than or equal to about 0.03, wherein R is reflectance and λ is wavelength, and wherein the cosmetic composition comprises a mixture of at least two colorants, wherein a first derivative of the spectrophotometric curve of each of the individual colorants does not exhibit (a), (b) and (c).

2. A cosmetic composition as defined by claim 1, wherein the composition comprises at least two discrete color domains containing at least one colorant each.

3. A cosmetic composition as defined by claim 2, wherein at least one of the color domains comprises a mixture of colorants.

4. A cosmetic composition as defined by claim 2, wherein at least one of the color domains comprises a carrier.

5. A cosmetic composition as defined by claim 2, wherein the color domains have an average size of from about 5 μm to about 500 μm.

6. A cosmetic composition as defined by claim 1, wherein the colorants, when solid or semisolid, have an average size of from about 0.01 μm to about 500 μm.

7. A cosmetic composition as defined by claim 1, comprising a physical mixture of the at least two colorants.

8. A cosmetic composition as defined by claim 1 wherein said mixture of at least two colorants is encapsulated.

9. A cosmetic composition as defined by claim 1, wherein the composition is in a form selected from the group consisting of powders, solid sticks, gels, lotions, creams and combinations thereof.

10. A cosmetic composition as defined by claim 1, wherein the composition is a personal care product.

11. A cosmetic composition as defined by claim 10, wherein the personal care product is selected from the group consisting of lipsticks, lip balms, makeup, foundations, blushes, face powders, mascaras, eyeliners, eye shadows, concealers, moisturizers, and skin cleansers.

12. A cosmetic composition as defined by claim 1, wherein the composition is adapted for delivery to a surface via a delivery mechanism selected from the group consisting of colloidal systems, delivery substrates, applicator pads, sponges, open cell foams, brushes, packages, mechanical spraying devices, electrostatic spraying devices, and printing devices.

13. A cosmetic composition adapted for application to a substrate to provide an applied cosmetic composition having a spectrophotometric curve, the applied cosmetic composition comprising at least two colorants; wherein a first derivative of the spectrophotometric curve comprises: a) a maximum peak in the region of from about 430 nm to about 520 nm occurs at a wavelength not greater than about 480 nm; b) a maximum peak in the region of from about 420 nm to about 650 nm occurs at a wavelength of from about 570 nm to about 630 nm; and c) a minimum valley in the region of from about 520 nm to about 580 nm has a Δ%R/Δλ of less than or equal to about 0.03, wherein R is reflectance and λ is wavelength, and wherein the first derivative of the spectrophotometric curve of each of the individual colorants does not exhibit (a), (b) and (c).

14. A cosmetic composition as defined by claim 13, wherein the composition comprises at least two discrete color domains containing at least one colorant each.

15. A cosmetic composition as defined by claim 13, wherein the composition is adapted for delivery to a surface via a delivery mechanism selected from the group consisting of colloidal systems, delivery substrates, applicator pads, sponges, open cell foams, brushes, packages, mechanical spraying devices, electrostatic spraying devices, and printing devices.

16. A cosmetic composition having a skin tone color and spectrophotometric curve, wherein a first derivative of the spectrophotometric curve comprises: a) a maximum peak in the region of from about 430 nm to about 520 nm occurs at a wavelength not greater than about 480 nm; b) a maximum peak in the region of from about 420 nm to about 650 nm occurs at a wavelength of from about 570 nm to about 630 nm; and c) a minimum valley in the region of from about 520 nm to about 580 nm has a Δ%R/Δλ of less than or equal to about 0.03, wherein R is reflectance and λ is wavelength, and wherein the cosmetic composition comprises a mixture of at least two colorants, wherein a first derivative of the spectrophotometric curve of each of the individual colorants does not exhibit (a), (b) and (c).

17. A cosmetic composition as defined by claim 16, wherein the composition comprises at least two discrete color domains containing at least one colorant each.

18. A cosmetic composition as defined by claim 17, wherein at least one of the color domains comprises a mixture of colorants.

19. A method for providing a cosmetic composition with a skin tone color, comprising adding at least two colorants to the cosmetic composition to provide the cosmetic composition with a first derivative of a spectrophotometric curve that comprises: a) a maximum peak in the region of from about 430 nm to about 520 nm occurs at a wavelength not greater than about 480 nm; b) a maximum peak in the region of from about 420 nm to about 650 nm occurs at a wavelength of from about 570 nm to about 630 nm; and c) a minimum valley in the region of from about 520 nm to about 580 nm has a Δ%R/Δλ of less than or equal to about 0.03, wherein R is reflectance and λ is wavelength, wherein a first derivative of a spectrophotometric curve of each of the individual colorants does not exhibit (a), (b) and (c).

20. The method of claim 19, wherein the composition is adapted for delivery to a surface via a delivery mechanism selected from the group consisting of colloidal systems, delivery substrates, applicator pads, sponges, open cell foams, brushes, packages, mechanical spraying devices, electrostatic spraying devices, and printing devices

21. A method of providing a colorant mixture for spectrophotometrically matching a cosmetic composition to a target substrate comprising:
a) determining a mixture of colorants adapted to provide a cosmetic composition having a spectrophotometric curve, wherein a first derivative of the spectrophotometric curve comprises: i) a maximum peak in the region of from about 430 nm to about 520 nm occurs at a wavelength not greater than about 480 nm; ii) a maximum peak in the region of from about 420 nm to about 650 nm occurs at a wavelength of from about 570 nm to about 630 nm; and iii) a minimum valley in the region of from about 520 nm to about 580 nm has a Δ%R/Δλ of less than or equal to about 0.03, wherein R is reflectance and λ is wavelength; and
b) admixing the colorants to form a mixture;
wherein a first derivative of a spectrophotometric curve of each of the individual colorants does not exhibit (a), (b) and (c).

22. A method of providing discrete color domains for spectrophotometrically matching a cosmetic composition to a target substrate comprising:
a) determining a combination of colorants adapted to provide a cosmetic composition having a spectrophotometric curve, wherein a first derivative of the spectrophotometric curvecomprises: i) a maximum peak in the region of from about 430 nm to about 520 nm occurs at a wavelength not greater than about 480 nm; ii) a maximum peak in the region of from about 420 nm to about 650 nm occurs at a wavelength of from about 570 nm to about 630 nm; and iii) a minimum valley in the region of from about 520 nm to about 580 nm has a Δ%R/Δλ of less than or equal to about 0.03, wherein R is reflectance and λ is wavelength; and
b) depositing the colorants into at least two discrete color domains;
wherein a first derivative of a spectrophotometric curve of each of the individual colorants does not exhibit (a), (b) and (c).
